(19) United States
(12) Patent Application Publication
(10) Pub. No.: US 2021/0100896 A1
(43) Pub. Date: Apr. 8, 2021

(54) METHODS AND COMPOSITIONS FOR STIMULATING THE IMMUNE SYSTEM

(71) Applicant: University of Miami, Miami, FL (US)

(72) Inventors: Eli GILBOA, Miami, FL (US); Brett SCHRAND, Miami, FL (US); Oliver MARTINEZ, Miami, FL (US); Agata LEVAY, Miami, FL (US)

(21) Appl. No.: 16/499,351

(22) PCT Filed: Apr. 3, 2018
(86) PCT No.: PCT/US18/25790

§ 371 Date: Sep. 30, 2019

Related U.S. Application Data
(60) Provisional application No. 62/481,720, filed on Apr. 5, 2017.

(51) Int. Cl. A61K 39/385; A61P 35/00

(52) U.S. Cl. CPC A61K 39/385; A61K 2039/6012; A61P 35/00

(57) ABSTRACT

The present invention relates, in part, to methods and compositions for enhancing anti-tumor immune responses. Particularly, the invention provides methods for enhancing the immune functions of Fc receptor (FcR)-expressing cells including dendritic cells, natural killer cells, macrophages, neutrophils, and eosinophils.

### CROSS-REFERENCE TO RELATED APPLICATION

This application claims the benefit of U.S. Provisional Application No. 62/481,720, filed Apr. 5, 2017, the contents of which are incorporated herein by reference in their entirety.

### FIELD

The present invention relates, in part, to methods and compositions for stimulating anti-tumor immune response. Particularly, the invention provides methods for enhancing the immune functions of Fc receptor (FcR)-expressing cells including dendritic cells, natural killer cells, macrophages, neutrophils, and eosinophils.

### BACKGROUND

Despite major advances in cancer treatment, cancer remains one of the leading causes of death globally. Hurdles in designing effective therapies include cancer immune evasion, in which cancer cells escape destructive immunity. Further still, many conventional cancer treatments such as radiation therapy and chemotherapy produce toxicity which significantly impacts a patient's ability to tolerate the therapy and/or impacts the efficacy of the treatment. Newer treatment modalities such as checkpoint inhibition provide the advantage of cell specificity that conventional cancer treatments lack. However, despite impressive patient responses, checkpoint inhibition therapy still fails in the overwhelming majority of patients.

An Fc receptor is a protein found on the surface of immune cells including dendritic cells, natural killer cells, macrophages, neutrophils, and eosinophils. Fc receptors bind to the Fc region of antibodies that are attached to, for example, infected cells or invading pathogens. Their activity stimulates phagocytic or cytotoxic cells to destroy infected cells or microbes by antibody-mediated phagocytosis or antibody-dependent cell-mediated cytotoxicity (ADCC).

Fc receptor (FcR)-expressing cells also play an important role in anti-tumor immunity. For example, dendritic cells are antigen-presenting cells which are activated upon recognition of tumor-associated molecules. Activated dendritic cells are capable of recruiting additional immune cells such as macrophages, eosinophils, natural killer cells and natural killer T cells. Dendritic cells also capture tumor antigens and present the antigens on their surfaces to activate T cells (e.g. cytotoxic T cells (CTLs)) which subsequently eliminate the tumor cells. Cancer cells can induce the dysfunction of dendritic cells thereby escaping immune destruction.

Additionally, ADCC may be an important mechanism of action of therapeutic monoclonal antibodies against tumor cells. ADCC is a mechanism of immune defense whereby an effector cell of the immune system actively lyses a target cell, whose membrane-surface antigens have been bound by specific antibodies. Classical ADCC is mediated by natural killer (NK) cells, macrophages, neutrophils, and eosinophils. In this regard, ADCC may be a key mechanism by which therapeutic monoclonal antibodies directed against cell surface targets on cancer cells exert their clinical effects.

Accordingly, novel methods that enhance the anti-tumor functions of Fc receptor (FcR)-expressing cells are beneficial for preventing and/or treating the onset, progression, or recurrence of cancers.

### SUMMARY

In various embodiments, the present invention provides improved methods for stimulating anti-tumor response mediated by dendritic cells including tumor infiltrating dendritic cells. The method comprises immunizing a subject against an antigen to generate an anti-antigen polyclonal antibody response, and administering to the subject a conjugate comprising the antigen linked to a tumor targeting ligand. In some embodiments, the antigen is a hapten such as dinitrophenol (DNP). Alternatively, the method may dispense with the initial immunizing step and comprise administering to the subject a conjugate comprising an antigen linked to a tumor targeting ligand, wherein the antigen is recognized by naturally occurring polyclonal antibodies already present in the subject. An exemplary antigen that is recognized by naturally occurring polyclonal antibodies is Galα1-3Galβ1-4GlcNAc-R.

In some embodiments, the present methods involve administering to the subject a conjugate comprising the antigen linked to a tumor targeting ligand. In some embodiments, the tumor targeting ligand comprises an oligonucleotide such as an aptamer. In other embodiments, the tumor targeting ligand comprises a protein-based agent such as an antibody, an antibody derivative, or a peptide. In an embodiment, the protein-based agent is a therapeutic monoclonal antibody. In some embodiments, the tumor targeting ligand recognizes one or more markers expressed on a tumor cell or the tumor environment. In some embodiments, the tumor targeting ligand recognizes a marker associated with non-transformed tumor endothelial cells or with the tumor vasculature, or a product upregulated in the tumor stroma. In exemplary embodiments, the tumor targeting ligand recognizes VEGF and/or osteopontin. In an illustrative embodiment, the tumor targeting ligand recognizes an immune checkpoint protein such as PD-1, PD-L1, PD-L2, or CTLA4.

In various embodiments, the methods of the invention are effective in treating cancers including, but not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In some embodiments, methods of the invention are combined with one or more therapies directed to increasing the neoantigenic content of tumor cells, blocking the function of the CD47 receptor, enhancing intratumoral DC infiltration, promoting intratumor immune infiltration by downregulation of β-catenin, promoting the survival and proliferation of tumor infiltrating T cells (e.g., by checkpoint blockade and/or tumor targeted costimulation), STING ligand administration, and local sublethal irradiation.

### BRIEF DESCRIPTION OF THE FIGURES

FIG. 1 provides a schematic of an exemplary method of coating tumor cells in situ with endogenous polyclonal antibodies.

FIG. 2 shows biodistribution of VEGF aptamer-DNP conjugate in 4T1 tumor bearing mice.

FIG. 3 shows that targeting DNP to palpable 4T1 tumors inhibited tumor growth in DNP immune mice.

FIG. 4 shows the role of immune subsets in VEGF-DNP mediated inhibition of tumor growth. The histograms, from left to right, represent untreated, VEGF-DNP+ISO, VEGF-DNP+CD4 Ab, VEGF-DNP+CD8 Ab, and VEGF-DNP+CD19 Ab.

FIG. 5 shows immunoglobulin deposit forms in the tumors of DNP, but not KLH, immunized mice treated with VEGF-DNP.

FIG. 6 shows combinatorial strategies potentiated tumor growth inhibition. The palpable 4T1 model was used as described in FIG. 2 with the exception that mice were treated only twice to reduce the effect of monotherapy.

FIG. 7 provides a schematic representation of an exemplary method to enhance the ADCC function of therapeutic monoclonal antibodies.

FIG. 8 shows enhancing the antitumor activity of a PD-L1 antibody by conjugating multiple DNP haptens to the antibody.

FIG. 9A-B shows a pair of graphs targeting of αGal trisaccharide to tumor cells. FIG. 9A shows the tumor volume following days' post transplantation. FIG. 9B shows the percentage of tumor free mice following days' post transplantation.

FIG. 10A-C are a series of confocal microscopy images showing VEGF-αGal mediated recruitment of antibodies from human serum to tumor biopsies. FIG. 10A shows renal cell cancer (RCC) and matched normal tissue. FIG. 10B shows Colon biopsy was incubated with VEGF-αGal or VEGF-DNP and 3 additional human sera and FIG. 100 shows Tumor biopsies from colon (shown in panel B), endomitrum, kidney (different from panel A), and melanoma were incubated with VEGF-αGal or VEGF-DNP conjugate and the human serum used in panel A.

### DETAILED DESCRIPTION

The present invention is based, in part, on the discovery that coating tumor cells with endogenous polyclonal antibodies is more effective in inducing anti-tumor immune response than coating tumors cells with monoclonal antibodies. For example, tumor cells which are “decorated” with such endogenous polyclonal antibodies are efficiently recognized and captured by tumor infiltrating dendritic cells. Accordingly, in various embodiments, the present methods significantly enhance anti-tumor immunity by stimulating dendritic cell functions.

The present invention is further based on the discovery that the ADCC functions of therapeutic antibodies may be enhanced by linking the antibodies to an antigen (e.g., a hapten). Particularly, therapeutic antibodies which recognize and target tumor cells are modified to include an antigen (e.g., a hapten). Such modified therapeutic antibodies are utilized to “decorate” tumor cells with the antigen which recruits polyclonal antibodies to the tumor cells. The polyclonal antibodies are in turn recognized by Fc receptor (FcR)-expressing cells including dendritic cells, natural killer cells, macrophages, neutrophils, and eosinophils which can destroy the coated tumor cells through ADCC and other mechanisms.

### Methods of Cancer Treatment

In various embodiments, the present invention provides methods for enhancing the anti-tumor activities of Fc receptor (FcR)-expressing cells including, but not limited to, dendritic cells, natural killer cells, macrophages, neutrophils, eosinophils, basophils, B lymphocytes, mast cells, and platelets.

In various embodiments, the present invention provides methods for stimulating dendritic cell functions by coating tumor cells with endogenous polyclonal antibodies.

The uptake of tumor antigens by tumor infiltrating dendritic cells (DC) is a key step in the induction of anti-tumor immunity. Specifically, the uptake of antibody coated tumor cells by DC via Fc receptors (FcRs) is an efficient antigen uptake mechanism leading to potent tumor immunity, in part because engagement of the FcRs on DC also promotes their maturation to become potent antigen presenting cells. There are, however, a number of limitations and challenges associated with the use of therapeutic monoclonal antibodies (mAbs) to promote antigen uptake and tumor immunity. These include antigen density, engagement of the inhibitory FcγRIIB receptor, and the monofunctionality of mAbs. Without wishing to be bound by theory, it is believed that coating tumor cells with endogenous polyclonal antibodies overcomes these limitations, thereby significantly enhancing dendritic cell-mediated anti-tumor immunity. For example, the present methods overcome the limitation associated with low density of the antigenic targets on tumor cells by use of haptens. Further, the present methods utilize pre-existing antibodies leading to the rapid coating of tumor cells with antibodies. In addition, coating tumor cells with different polyclonal IgG subtypes, IgA, and/or IgE antibodies may have a combinatorial and synergistic effect on killing tumor cells as well as DC uptake and/or maturation.

In various embodiments, coating the tumor cells with endogenous polyclonal antibodies also stimulates the anti-tumor activities of Fc receptor (FcR)-expressing cells other than dendritic cells such as natural killer cells, macrophages, neutrophils, eosinophils, basophils, B lymphocytes, mast cells, and platelets. In an embodiment, the present methods enhance the destruction of tumor cells through ADCC as mediated, for example, by natural killer cells.

In various embodiments, the present methods comprise the steps of first immunizing a patient against an antigen (e.g., a hapten) to generate an anti-antigen polyclonal antibody response. Next, tumor cells are coated with the antigen (e.g., hapten) by administering to the patient a conjugate comprising an antigen (e.g., a hapten) linked to a tumor targeting ligand which will attract the preexisting anti-antigen (e.g., anti-hapten) polyclonal antibodies to the tumor cells. This, in turn, enhances the uptake of the antibody coated tumor cells by tumor resident DC in an Fc:FcR mediated manner. Further, engagement of the FcRs on DCs also induces DC maturation, leading to stimulation of a potent T cell response against the tumor cells.

In various embodiments, the present methods include an initial vaccination step in which a patient is immunized with an antigen (e.g., a hapten) to generate an anti-antigen polyclonal antibody response. In various embodiments, any antigen known in the art may be utilized for the present invention. In this regard, a suitable antigen can be determined by a person in the art. In exemplary embodiments, the antigen may be an influenza or a Measles, Mumps, and Rubella Virus (MMR) antigen.

In various embodiments, the antigen is a hapten. As used herein, a hapten refers to small molecules that elicit an immune response only when attached to a large carrier such as a protein. Haptens include hormones, tumor markers and viral proteins among others. For example, haptens may include molecules with a steroid backbone that are selected from the group comprising sterols, bile acids, sexual hormones, corticoids, cardenolides, cardenolide-glycosides, bufadienolides, steroid-sapogenines and steroid alkaloids. These haptens are capable of binding to a specific receptor, e.g. to antibodies or antibody fragments which are directed against the hapten. In some embodiments, the hapten is selected from the group comprising cardenolides and cardenolide-glycosides. Representatives of these substance classes are digoxigenin, digitoxigenin, gitoxigenin, strophanthidin, digoxin, digitoxin, ditoxin and strophanthin. Other exemplary haptens include, but are not limited to dinitrophenol (DNP), fluorescein, and biotin. In an embodiment, the hapten is DNP.

In additional and/or alternative embodiments, the first vaccination step of immunizing a patient against an antigen (e.g., a hapten) may be dispensed through exploitation of naturally occurring polyclonal antibodies in the patient. In an embodiment, the present methods utilize naturally occurring polyclonal antibodies specific to the hapten Galα1-3Galβ1-4GlcNAc-R.

In various embodiments, the present methods comprise the step of administering to a patient a conjugate comprising an antigen (e.g., a hapten) linked to a tumor targeting ligand. In some embodiments, this step coats the tumor cells with the antigen (e.g., hapten) and attracts any existing anti-antigen (e.g., anti-hapten) polyclonal antibodies to the tumor cells. In various embodiments, the conjugate may comprise any antigen (e.g., hapten) as described herein.

In various embodiments, the conjugate comprises a tumor targeting ligand that may target any markers expressed on tumor cells or the tumor microenvironment. Tumor specific markers are known in the art, and may include, but are not limited to, MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD20, CD22, CD30, CD33, CD37, CD56, CD70, CD74, CD138, AGS16, MUC1, GPNMB, Ep-CAM, PD-L1, PD-L2, 4-1BB, TIM-3, PMSA, and BCMA (TNFRSF17). In some embodiments, the tumor targeting ligand may be directed to a marker associated with non-transformed tumor endothelial cells or with the tumor vasculature, or a product upregulated in the tumor stroma. In exemplary embodiments, the tumor targeting ligand is directed to VEGF and/or osteopontin (OPN).

In various embodiments, the tumor targeting ligand of the invention may be in any format or be any molecule capable of binding to a marker (e.g., a tumor marker).

In some embodiments, the tumor targeting ligand comprises an oligonucleotide, such as DNA or RNA. In an exemplary embodiment, the tumor targeting ligand comprises an oligonucleotide aptamer. In an embodiment, the tumor targeting ligand comprises a VEGF aptamer. In another embodiment, the tumor targeting ligand comprises an OPN aptamer.

In some embodiments, the oligonucleotide molecule (e.g., an aptamer) has one or more nucleotide substitutions (e.g. at least one of adenine, guanine, thymine, cytosine, uracil, purine, xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynylcytosine, 5-fluorouracil, 5-bromouracil, pseudoisocytosine, 2-hydroxy-5-methyl-4-triazolopyridin, isocytosine, isoguanin, inosine, non-naturally occurring nucleobases, locked nucleic acids (LNA), peptide nucleic acids (PNA), variants, mutants, analogs or combinations thereof.

In various embodiments, the oligonucleotide molecule (e.g., an aptamer) comprises fluoro-modified pyrimidines, e.g. 2′-fluoro-modified pyrimidines, e.g. one or more of 2′-fluoro-cytosine (C), 2′-fluoro-thymine (T), and 2′-fluorouracil (U).

In some embodiments, the tumor targeting ligand comprises a protein-based targeting agent. In an exemplary embodiment, the protein-based targeting agent is an antibody, antibody format, or paratope-comprising fragment thereof directed against a marker (e.g., a tumor marker). In various embodiments, the antibody is a full-length multimeric protein that includes two heavy chains and two light chains. Each heavy chain includes one variable region (e.g., V_{H}) and at least three constant regions (e.g., CH₁, CH₂ and CH₃), and each light chain includes one variable region (V_{L}) and one constant region (C_{L}). The variable regions determine the specificity of the antibody. Each variable region comprises three hypervariable regions also known as complementarity determining regions (CDRs) flanked by four relatively conserved framework regions (FRs). The three CDRs, referred to as CDR1, CDR2, and CDR3, contribute to the antibody binding specificity. In some embodiments, the antibody is a chimeric antibody. In some embodiments, the antibody is a humanized antibody.

In some embodiments, the protein-based targeting agent is an antibody derivative or format. In some embodiments, the protein-based targeting agent comprises a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; a peptide aptamer; an alterases; a plastic antibodies; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, a F(ab′)₂, a peptide mimetic molecule, or a synthetic molecule, as described in U.S. Pat. No. 7,417,130, US 2004/132094, U.S. Pat. No. 5,831,012, US 2004/023334, U.S. Pat. Nos. 7,250,297, 6,818,418, US 2004/209243, U.S. Pat. Nos. 7,838,629, 7,186,524, 6,004,746, 5,475,096, US 2004/146938, US 2004/157209, U.S. Pat. Nos. 6,994,982, 6,794,144, US 2010/239633, U.S. Pat. No. 7,803,907, US 2010/119446, and/or U.S. Pat. No. 7,166,697, the contents of which are hereby incorporated by reference in their entireties. See also, Storz MAbs. 2011 May-June; 3(3): 310-317.

In various embodiments, the protein-based targeting agent is a therapeutic antibody that recognizes one or more markers on tumor cells or in the tumor microenvironment (e.g., tumor stroma). In such embodiments, the therapeutic antibodies are linked to an antigen (e.g., a hapten) and functions as a tumor targeting ligand to coat the targeted tumor cells with the antigen (e.g., hapten). The “decorated” tumor cells attracts existing anti-antigen (e.g., anti-hapten) polyclonal antibodies to the tumor cells which then activates the anti-tumor actions of Fc receptor (FcR)-expressing cells such as dendritic cells, natural killer cells, macrophages, neutrophils, and eosinophils. For example, the “decorated” tumor cells may be destroyed by natural killer cells via ADCC. Accordingly, in an embodiment, methods of the invention enhance the ADCC functions associated with therapeutic antibodies which recognize and target tumor cells.

A therapeutic antibody can be linked to an antigen (e.g., a hapten) using methods known in the art. For example, a therapeutic antibody can be conjugated to the antigen using chemical conjugation techniques. In another example, a therapeutic antibody can be linked to the antigen using recombinant DNA techniques. In some embodiments, the therapeutic antibody is linked to the antigen through linkers. In various embodiments, linking the therapeutic antibody to the antigen does not interfere with the antibody's ability to recognize and bind to a marker associated with tumor cells or the tumor microenvironment (e.g., tumor stroma).

Any therapeutic antibodies that recognize and target tumor cells can be utilized in the invention. Exemplary therapeutic antibodies include, but are not limited to, anti-CD20 antibodies (e.g., Rituximab), anti-Her2 antibodies (e.g., Trastuzamab), and anti-EGFR antibodies (e.g., Cetuximab). Additional therapeutic antibodies that may be utilized include antibodies that target immune checkpoint molecules which are described elsewhere herein. In an embodiment, the therapeutic antibody targets PD-1, PD-L1, PD-L2, and/or CTLA4.

In some embodiments, the protein-based targeting agent is a peptide directed to a marker (e.g., a tumor marker).

In various embodiments, the tumor targeting ligand binds one or more of these markers (e.g., tumor markers). In some embodiments, the tumor targeting ligand is monovalent and binds a marker (e.g., a marker expressed on tumor cells or the tumor microenvironment). In some embodiments, the tumor targeting ligand is multivalent and binds to one or more markers (e.g., markers expressed on tumor cells or the tumor microenvironment). In an embodiment, the tumor targeting ligand is multivalent and binds to both VEGF and OPN on tumor cells. In some embodiments, the tumor targeting ligand is a monomer. In some embodiments, the tumor targeting ligand is multimeric.

### Pharmaceutical Compositions and Administration

The present invention provides pharmaceutical compositions comprising one or more antigens (e.g., haptens) as described herein. The present invention further provides pharmaceutical compositions comprising one or more conjugates comprising an antigen linked to a tumor targeting ligand as described herein. The In some embodiments, the pharmaceutical compositions described herein are in the form of a pharmaceutically acceptable salt.

The antigens (e.g., haptens) described herein can possess a sufficiently basic functional group, which can react with an inorganic or organic acid, or a carboxyl group, which can react with an inorganic or organic base, to form a pharmaceutically acceptable salt. Similarly the conjugate comprising an antigen (e.g., a hapten) linked to a tumor-targeting ligand can possess a sufficiently basic functional group, which can react with an inorganic or organic acid, or a carboxyl group, which can react with an inorganic or organic base, to form a pharmaceutically acceptable salt.

A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

Pharmaceutically acceptable salts include, by way of non-limiting example, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, and tartarate salts.

The term “pharmaceutically acceptable salt” also refers to a salt of the compositions of the present invention having an acidic functional group, such as a carboxylic acid functional group, and a base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

Any pharmaceutical compositions described herein can be administered to a subject as a component of a composition that comprises a pharmaceutically acceptable carrier or vehicle. Such compositions can optionally comprise a suitable amount of a pharmaceutically acceptable excipient so as to provide the form for the proper routes of administration.

In various embodiments, routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection.

Any pharmaceutical composition described herein can be administered parenterally. Such pharmaceutical composition can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

### Combination Therapy and Additional Therapeutic Agents

In various embodiments, the present invention relates to combination therapy. In various embodiments, the combination therapy may result in synergistic anti-cancer effects, for example, in reducing the likelihood of cancer onset, progression, and/or recurrence.

In various embodiments, the present invention relates to combination therapy with one or more therapies directed to increasing the neoantigenic content of the tumor cells, blocking the function of the “don't-eat-me” CD47 receptor, enhancing intratumoral DC infiltration, promoting intratumor immune infiltration by downregulation of β-catenin, promote the survival and proliferation of tumor infiltrating T cells (e.g., by checkpoint blockade and/or tumor targeted costimulation), STING ligand administration, and local sublethal irradiation.

In some embodiments, the present invention pertains to combination therapy with an approach that increases the neoantigenic content of the tumor cells. In some embodiments, the approach involves inhibition and/or downregulation of key mediators of antigen processing pathways. In some embodiments, the approach involves inhibition and/or downregulation of ERAAP. In some embodiments, the approach involves inhibition and/or downregulation of transporter associated with antigen processing (TAP). In some embodiments, the approach involves inhibition and/or downregulation of invariant chain (Ii).

ERAAP is an ER-resident aminopeptidase that trims the TAP-transported peptides to optimize their association with the nascent MHC class I molecules (see Nature. 2002; 419(6906):480-3). Importantly, without wishing to be bound by theory, ERAAP deficiency induces significant alterations in the MHC class I presented peptidome. Some peptides are lost while new peptides appear, the latter probably, without wishing to be bound by theory, because they escape ERAAP processing. Like TAP-deficient cells, ERAAP-deficient cells are immunogenic in wild type mice inducing T cell response against the new ERAAP-loss induced peptides to which the wild type mouse has not been tolerized, and inhibit tumor growth. The new peptides are presented both by classical MHC class Ia molecules as well as by nonclassical MHC class Ib molecules, specifically Qa-1b. A dominant peptide presented by Qa-1b in the H-2b background was identified as FYAEATPML (FL9) derived from FAM49B protein). Qa-1b restricted presentation of the FL9 peptide stimulates CD8+ T cell responses in wild type mice that can kill ERAAP-deficient, but not ERAAP sufficient, targets.

TAP is a critical component of MHC class I presentation responsible for transporting the proteasome generated peptides from the cytoplasm to the ER where they are loaded onto the nascent MHC class I molecules (see Nat Rev Immunol. 2011; 11(12):823-36.) TAP function is frequently downregulated in tumors conceivably, without wishing to be bound by theory, to avoid immune recognition. TAP-deficient cells present novel peptide-MHC complexes resulting from alternative antigen processing pathways that are upregulated or become dominant in the absence of the canonical TAP-mediated pathway. TAP deficiency-induced peptides, referred to as “T cell epitopes associated with impaired peptide processing” or TEIPP, are presented by classical MHC class Ia molecules as well as by nonclassical Qa-1b molecules. Importantly, TAP-deficient cells or DC loaded with TEIPP peptide restricted to both the classical MHC Ia and Qa-1b can stimulate CD8+ T cell responses in wild type mice and vaccination with TEIPP loaded DC, TAP-deficient DC, or adoptive transfer of TEIPP specific CD8+ T cells was shown to inhibit the growth of TAP-deficient, but not TAP sufficient, tumors.

Invariant chain is a polypeptide involved in the formation and transport of MHC class II protein. The cell surface form of the invariant chain is known as CD74. MHC class II's path toward the cell surface involves, in the rough endoplasmic reticulum, an association between the alpha and beta chains and a Ii, which stabilizes the complex. Without the invariant chain, the alpha and beta proteins will not associate. Ii trimerizes in the ER, associates with MHC class II molecules and is released from the ER as a nine subunit complex. This MHC-invariant complex passes from the RER to, and out of, the Golgi body. Before moving to the cell surface, the vesicle containing this complex fuses with an endocytic compartment where an external protein has been broken into fragments. Here the invariant chain is proteolytically degraded and a peptide from the external protein associates with the MHC II molecule in the channel between the alpha-1 and beta-1 domains. The resulting MHC II-peptide complex proceeds to the surface where it is expressed.

In some embodiments, the approach involves inhibition and/or downregulation of a nonsense-mediated mRNA (NMD) process. NMD is an evolutionarily conserved surveillance mechanism in eukaryotic cells that prevents the expression of mRNAs containing a premature termination codon (PTC). In some embodiments, the approach involves the use of an inhibitor such as a small interfering RNA (siRNA) which downregulates certain NMD factors (e.g. SMG1, UPF1, UPF2, UPF3, RENT1, RENT2, elF4A, UPF1, UPF2, UPF3B, RNPS1, Y14, MAGOH, NMD1, or combinations thereof).

In some embodiments, the present invention pertains to combination therapy with an approach that enhances the intratumoral accumulation of proinflammatory immune cells. In some embodiments, such an approach may involve the downregulation of β-catenin. In some embodiments, downregulation of β-catenin may be achieved through the use of nucleolin or EpCAM aptamer-siRNA conjugates. In other embodiments, the present invention pertains to combination therapy with STING ligand administration, and/or irradiation such as local sublethal irradiation, so as to promote intratumoral immune infiltration.

In some embodiments, the present invention pertains to combination therapy with an approach that increases intratumoral DC infiltration. In some embodiments, the approach increases the number of CD141+DC cells in the tumor or tumor microenvironment. In an embodiment, the approach involves administration of Flt3 ligand (Flt3L) to a subject.

In some embodiments, the present invention pertains to combination therapy with an approach that blocks the function of the CD47 receptor. CD47 is an inhibitory receptor that prevents phagocytic uptake of CD47 expressing cells. CD47 is upregulated on many tumors. In some embodiments, the approach involves the use of anti-CD47 antibodies which blocks CD47 function including a CD47 depleting antibody.

In some embodiments, the present invention pertains to combination therapy with an approach that enhances the survival and proliferative capacity of tumor infiltrating T cells. The approach may involve blocking inhibitory receptors and engaging stimulatory receptors. Accordingly, in some embodiments, the present invention relates to combination therapy with one or more immune-modulating agents, for example, without limitation, agents that modulate immune checkpoint. In various embodiments, the immune-modulating agent targets one or more of PD-1, PD-L1, and PD-L2. For instance, in some embodiments, the immune-modulating agent is an antibody such as, by way of non-limitation, nivolumab, (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), MPDL328OA (ROCHE). In some embodiments, the immune-modulating agent is an agent that targets one or more of CTLA-4, AP2M1, CD80, CD86, SHP-2, and PPP2R5A. In various embodiments, the immune-modulating agent is an antibody specific for one or more of CTLA-4, AP2M1, CD80, CD86, SHP-2, and PPP2R5A. For instance, in some embodiments, the immune-modulating agent is an antibody such as, by way of non-limitation, ipilimumab (MDX-010, MDX-101, Yervoy, BMS) and/or tremelimumab (Pfizer). In some embodiments, the immune-modulating agent targets one or more of CD137 (4-1BB) or CD137L. In various embodiments, the immune-modulating agent is an antibody specific for one or more of CD137 (4-1BB) or CD137L. For instance, in some embodiments, the immune-modulating agent is an antibody such as, by way of non-limitation, urelumab (also known as BMS-663513 and anti-4-1BB antibody). In various embodiments, the antibodies stimulate 4-1BB mediated co-stimulation.

Additional immune approaches that may be used in the combination therapies as described herein include, but are not limited to, Treg elimination, IDO inhibition, LIGHT, OX40, GITR stimulation, STING ligand and IFNαinduction.

In some embodiments, the present invention pertains to chemotherapeutic agents as additional therapeutic agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE, vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

### Methods of Treatment

In some embodiments, the present invention relates to the treatment of, or a patient having cancer. As used herein, cancer refers to any uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

Illustrative cancers that may be treated include, but are not limited to, carcinomas, e.g. various subtypes, including, for example, adenocarcinoma, basal cell carcinoma, squamous cell carcinoma, and transitional cell carcinoma), sarcomas (including, for example, bone and soft tissue), leukemias (including, for example, acute myeloid, acute lymphoblastic, chronic myeloid, chronic lymphocytic, and hairy cell), lymphomas and myelomas (including, for example, Hodgkin and non-Hodgkin lymphomas, light chain, non-secretory, MGUS, and plasmacytomas), and central nervous system cancers (including, for example, brain (e.g. gliomas (e.g. astrocytoma, oligodendroglioma, and ependymoma), meningioma, pituitary adenoma, and neuromas, and spinal cord tumors (e.g. meningiomas and neurofibroma).

Illustrative cancers that may be treated include, but are not limited to, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In various embodiments, the present methods induce and/or enhance anti-tumor immune responses mediated by infiltrating dendritic cells. In some embodiments, the present methods induce and/or enhance anti-tumor responses mediated by CD4+ T cells. In various embodiments, the present methods induce and/or enhance humoral immune responses against tumors. In various embodiments, the present methods induce and/or enhance ADCC as mediated by Fc receptor (FcR)-expressing cells including dendritic cells, natural killer cells, macrophages, neutrophils, and eosinophils. In various embodiments, the present invention induces and/or enhances the destruction of immune suppressive cells. In some embodiments, the present invention induces and/or enhances the destruction of tumor cells.

### Kits

The invention also provides kits for the administration of any agent described herein (e.g. a hapten or a conjugate comprising a hapten linked to a tumor-targeting ligand). The kit is an assemblage of materials or components, including at least one of the inventive pharmaceutical compositions described herein. Thus, in some embodiments, the kit contains at least one of the pharmaceutical compositions described herein.

The exact nature of the components configured in the kit depends on its intended purpose. In one embodiment, the kit is configured for the purpose of treating human subjects.

Instructions for use may be included in the kit. Instructions for use typically include a tangible expression describing the technique to be employed in using the components of the kit to effect a desired outcome, such as to treat cancer. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials and components assembled in the kit can be provided to the practitioner stored in any convenience and suitable ways that preserve their operability and utility. For example, the components can be provided at room, refrigerated or frozen temperatures. The components are typically contained in suitable packaging materials. In various embodiments, the packaging material is constructed by well-known methods, preferably to provide a sterile, contaminant-free environment. The packaging material may have an external label which indicates the contents and/or purpose of the kit and/or its components.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein. This invention is further illustrated by the following non-limiting examples.

### Examples

### Example 1: Methods to Coat Tumor Cells in DNP Immune Mice with Polyclonal DNP-Specific Antibodies

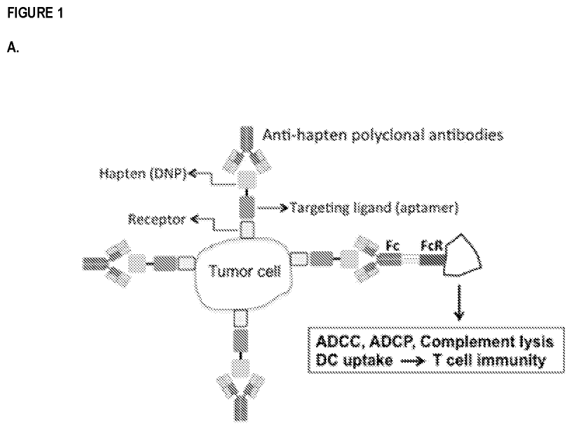

A simple, broadly applicable, and cost-effective approach was developed to coat tumor cells with endogenous preexisting polyclonal antibodies that elicit a strong T-cell dependent vaccinal effect leading to tumor rejection. Without wishing to be bound by theory, it is believed that the approach of coating tumor cells with endogenous polyclonal antibodies is superior to coating tumor cells with specific monoclonal antibodies. The approach is illustrated in FIG. 1, which is non-limiting, and includes the following steps:
I. First, the patient (or experimental subject) is vaccinated against a harmless or beneficial antigen (e.g., influenza or MMR antigens), or a simple hapten like dinitrophenol (DNP), to generate a polyclonal antibody response. Alternatively, preexisting naturally occurring polyclonal antibodies may be exploited so as to eliminate the vaccination step altogether. This can be achieved by vaccinating the patients against antigens recognized by naturally occurring antibodies.
II. In parallel, the hapten or antigen is conjugated to a ligand that will target the hapten/antigen to the tumor lesion, namely a ligand that binds a tumor cell specific product or to products expressed in the tumor stroma. Ligands can be antibodies, peptides, or oligonucleotide aptamers. In an exemplary embodiment, the ligand is an aptamer.
III. Next, the ligand-hapten conjugate is injected systemically into tumor bearing patient (or mouse). Without wishing to be bound by theory, it is believed that the ligand targets the hapten/antigens to the tumor cells thereby “decorating” the tumor cells or alternatively components within the tumor stroma with the hapten. This in turn attracts and opsonizes the tumor cells or the tumor stromal elements with the preexisting hapten-specific polyclonal antibodies, thus recapitulating the scenario of miHC mismatch transplanted tumor cells. In this regard, the efficient rejection of minor histocompatibility (MiHC) mismatched tumors, essentially syngeneic tumors expressing foreign antigens on the cell surface, is mediated by pre-existing polyclonal antibodies present in a subject (e.g., a mouse) that recognize the mismatched targets on the implanted tumor cells, thereby enhancing their uptake by dendritic cell (DC) and stimulating a protective T cell response. It is believed that the antibody-mediated rejection of the MiHC mismatched tumors is significantly more potent than that of tumor cells coated with specific monoclonal antibodies.

### Development of VEGF- and OPN-DNP Conjugates

To further develop the approach as outlined above, oligonucleotide aptamers to VEGF or osteopontin (OPN) were used to target dinitrophenol (DNP) to tumors stroma in situ. In this regard, oligonucleotide aptamer offer potentially significant advantages in terms of development, manufacture, cost, and conjugation.

Reflecting increased angiogenic activity of tumors, VEGF is expressed at elevated levels in tumors compared to normal tissues or the circulation. In Balb/c mice, circulating levels of VEGF are barely detectable by ELISA (e.g., less than 5 pg/ml) and are not increased in tumor bearing mice even after irradiation. VEGF and OPN aptamers can home to and accumulate primarily in the tumor tissue but not normal tissue, and can be used to target specific aptamers to tumor in mice in induce effective anti-tumor immunity while avoiding toxicities associated with systemic administration.

The receptors for both OPN and VEGF are upregulated on activated endothelial cells in the tumor vasculature. The main receptors for OPN are various integrins, i.e., αvβ1, αvβ3, αvβ5, and CD44. The main receptors for VEGF are VEGFR1/KDR/Flk1, VEGFR2/Flt1, and Neuropilin-(NRP1). The integrin receptors are also expressed on tumor cells, and many tumor cells express at least one of the two VEGF receptors, most often VEGFR1. VEGF also binds weakly to heparin and is retained on the surface of tumor cells via binding to heparan sulfate containing proteoglycans. Indeed, VEGF isoforms were shown to bind to tumor cells that in colorectal cancer patient correlated with disease progression.

Without wishing to be bound by theory, it is believed that VEGF or OPN which are secreted into the tumor stroma will bind to their receptors on the surface of the activated tumor endothelial cells and tumor cells. Thus the VEGF or OPN binding aptamers (providing they do not compete with the cognate receptors or heparan sulfate) are capable of targeting the conjugated cargo like DNP to tumor cells and/or the tumor vasculature, inducing an anti-tumor and/or an anti-activated endothelial immune response, respectively. This in turn may lead to protective anti-tumor immunity. Indeed, immunizing against nontransformed tumor endothelial cells, or products upregulated in the tumor stroma that are not expressed in the transformed tumor cells, can induce potent anti-tumor immunity in the absence of detectable autoimmune pathology.

A short 20-nt oligonucleotide was chemically conjugated to DNP and hybridized to a VEGF or OPN aptamer extended at the 3′ end with a complementary oligonucleotide. The conjugates bound to plate immobilized VEGF or OPN and could be stained with DNP specific antibodies. Biodistribution of VEGF-DNP conjugates in tumor bearing mice was evaluated by systemic administration of ³²P-labeled conjugates via tail vein injection. Specifically, Balb/c mice were implanted subcutaneously with 4T1 tumor cells and when tumors reached a diameter of −3 mm ³²P-labeled VEGF aptamer-DNP conjugate was injected via the tail vein. 18 hours later mice were sacrificed, organs were isolated, partially perfused by incubation for 30 minutes in PBS at room temperature, and radioactivity counted.

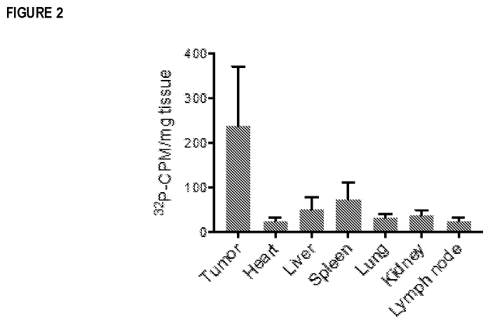

As shown in FIG. 2, at 18 hours post administration, the VEGF-DNP conjugates accumulated preferentially in the subcutaneously implanted 4T1 tumors, reflecting the preferential expression of VEGF in tumor tissue compared to normal tissue. The low levels of VEGF-DNP conjugates seen in normal tissue correspond mostly to conjugate in the blood that could be further reduced by more extensive perfusion.

### Inhibition of Tumor Growth in 4T1 Tumor Bearing DNP-Immune Mice

In the following experiments, Balb/c mice immunized with DNP-KLH or KLH alone were implanted subcutaneously with 4T1 breast carcinoma tumor cells and when tumors became palpable, the tumor bearing mice were treated with VEGF- or OPN-DNP conjugates. Tumor growth was monitored.

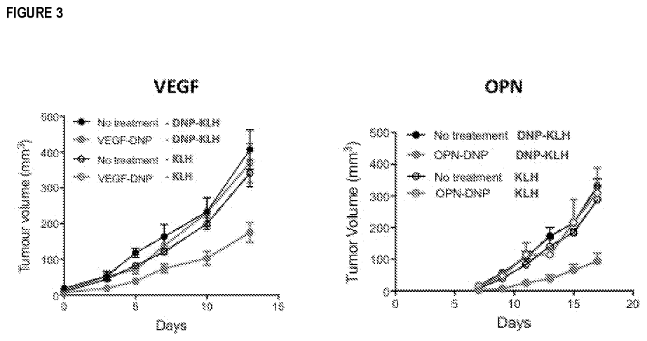

Specifically, the mice were immunized for three times at two weeks apart with DNP-KLH or KLH. At 10 days post last vaccination, 4T1 tumor cells were injected subcutaneously. About 7-8 days later when tumors became palpable VEGF- or OPN-DNP conjugates were administered systemically twice at 3 days apart by tail vein injection and tumor growth was monitored. As shown in FIG. 3, both VEGF- and OPN-targeted DNP conjugates inhibited tumor growth in DNP immune (DNP-KLH), but not DNP-naïve (KLH) vaccinated mice.

The 4T1 breast carcinoma tumor cell line is a poorly immunogenic and notoriously difficult to treat tumor. Most immune based monotherapies are ineffective against palpable 4T1 tumors (in contrast to that of palpable immunogenic tumors like CT26, MC38 or A20). Given that the vaccination conditions (two treatments) were probably suboptimal, these observation indicate that the approach as described herein was potent. These experiments also showed that clearance of the aptamer-DNP immunocomplexes by the reticuloendothelial system prior to reaching the tumor was not a limiting factor.

### Example 2: Additional Improvement of Methods to Coat Tumor Cells in DNP Immune Mice with Polyclonal DNP-Specific Antibodies

Methods were developed to enhance the titer, persistence, and avidity of the vaccine-induced polyclonal antibody response. Specifically, two main strategies are used. The first strategy involved costimulating the BAFF or APRIL receptors using DNA plasmid encoded trivalent ligands. Additionally, the second strategy involved increasing the titers of IgE responses, albeit to limited extent to avoid anaphylactic shock, by controlled dosing with systemically administered IL-4, IL-5 or IL-13.

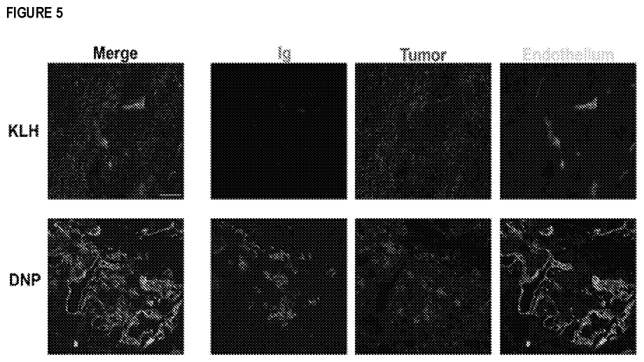

Pharmacokinetics and tissue distribution of intravenously injected aptamer-DNP conjugates are determined in DNP naïve tumor bearing mice by staining tumor and tissue sections with anti-DNP antibodies or ³²P-labeled conjugates. Preliminary experiments have demonstrated the preferential accumulation of VEGF-DNP conjugates in the tumor compared to normal tissues and organs (see FIG. 2). Immunohistochemical analysis is expected to reveal close association of the VEGF or OPN aptamer-DNP conjugate with tumor cells (identified by H&E staining) and endothelial cells expressing the corresponding receptors. Association with tumor endothelial cells is analyzed by high resolution confocal microscopy and staining endothelial cells with CD31 and/or von Willebrand antibodies. Intravenously injected aptamer conjugate is expected to exhibit a circulation half-life of 18-48 hours and accumulate in the tumor rapidly, within 2-6 hours after injection. The larger multivalent conjugates may be less able to penetrate deep into the tumor tissue and bind to tumor cells. This could negatively impact their anti-tumor activity except if their primary mechanism of action is mediated by binding to the tumor vasculature. The intratumoral and tissue distribution of immunoglobulin antibodies (Ig) are evaluated using control KLH and DNP-KLH immunized mice as described elsewhere (see FIG. 5). It is expected that administration of conjugates to DNP-KLH, immunized mice, but not KLH immunized mice, would enhance Ig accumulation in the tumor that will parallel the distribution of the aptamer-DNP conjugates in KLH immunized mice.

The tamoxifen-induced BRAF mutant melanoma and autochthonous PDA models are used to test the inhibition of tumor growth by VEGF and OPN aptamer-DNP conjugates. Specifically, the conjugates are first screened in a subcutaneously implanted palpable 4T1 model. The best performing aptamer conjugates are then tested in the tamoxifen induced BRAF mutant melanoma and KPC derived autochthonous PDA models.

For the tamoxifen-induced BRAF/PTEN model, two strains of mice are mated and F1 mice are induced with hydroxytamoxifen applied to the skin. Tumors develop locally within 3-4 weeks with 100% penetrance and subsequently metastasize to the ear and base of tail. The mice can be followed in real time, and the inguinal lymph nodes and lung can be evaluated post mortem. Treatment with aptamer conjugates is initiated by intravenous injections when the local tumors reach about 3 mm in height. Survival is used as main endpoint.

The ability of the VEGF and OPN aptamer-DNP conjugates to inhibit tumor is also evaluated using the autochthonous PDA model. The autochthonous PDA model is a pancreatic cancer model in which 3 mm tumor fragments from KPC mice are implanted surgically into the pancreas of wild type C57BU6 mice. Tumors develop synchronously in all mice recapitulating the intense desmoplasia and leukocytic infiltration seen in tumors arising in genetically engineered KPC mice. Tumors are histologically detectable after 4 weeks and mice develop morbidity requiring euthanization after about 10-12 weeks. In this model treatment with aptamer conjugates starts week 3-4 after tumor implantation. Progressive weight loss and survival are used as main endpoints. Only the most effective strategies will be tested in the spontaneous KPC model.

Additional tumor models that may be used include the 4T1 breast carcinoma post-surgical lung metastasis and/or the MCA carcinogen-induced fibrosarcoma model.

### Example 3: Mechanistic Studies

Mechanistic studies are carried out to test whether tumor targeted coating with polyclonal antibodies are taken up by tumor resident DC to elicit a superior T cell mediated adaptive anti-tumor response. Alternative or contributory mechanisms such as a direct antiangiogenic effect via ADCC mediated killing of tumor vascular endothelial cells are also evaluated. Studies are carried out in the 4T1 tumor model using DNP immune and control (KLH vaccinated) mice treated systemically with the VEGF-DNP conjugates. Analysis is carried out to measure impact of intervention on tumor growth, immune responses, and intratumoral antibody and conjugate accumulation.

The role of humoral responses and Fc-FcR interaction is determined by (i) antibody depletion of the DNP immune mice using CD19 Ab and transfer of serum to naïve mice, leading to loss or gain of protective anti-tumor immunity, respectively; and (ii) loss of tumor inhibition in FcγRI, FcγRIII, FcεRI deficient mice. Complement activation at the tumor site is measured by immunohistochemistry using an antibody that either targets terminal membrane attack complex (anti-05b-9) or opsonin product C3b. Alternatively, these antibodies are used for western blot and ELISA of tumor lysates. If activated complement within tumors is not observed, expression of membrane-bound complement regulators such as CD46 and CD59 on tumor cells and infiltrating immune cells are determined by histology and/or flow cytometry to analyze whether complement has being cleared or blocked from activation.

The role of adaptive T cell immunity is determined by (i) antibody depletion of CD4 and/or CD8 cells; (ii) adoptive transfer of T cell subsets from conjugate treated tumor bearing DNP immune to naïve mice that will be challenged with tumor; and (iii) using an immunogenic model like A20 lymphoma or CT26 colon carcinoma model whereby monotherapy with VEGF or OPN-DNP conjugates is likely to induce long term regression, challenge the cured mice with same and irrelevant tumor to determine if treatment has elicited tumor-specific immunological memory.

The role of NK and/or macrophages mediated direct killing of antibody coated tumor cells is evaluated by Ab depletion of NK cells and macrophages with clondronate liposomes.

Additional evidence for the role of innate and adaptive immunity is sought by measuring the composition of the intratumoral immune infiltrate using multicolor flow cytometry, including but not limited to: CD4 and CD8 T cells, Treg (CD4+Foxp3+), MDSC (CD11b+Gr1+), tumor cross-presenting BATF3+DC (CD11c+Flt3+CD103+CCR7+), tumor resident memory T cells (CD8+CD69+CD62L-CCR7-), exhausted T cells expressing any combination of PD-1, Tim3, and/or LAG3, and the presence of polyfunctional CD4 and CD8 T cells expressing IFN, IL-2 and TNF which correlate with protective immunity. It is expected that in the conjugate treated tumor bearing DNP immune, but not DNP naïve, mice, there is a significant enhancement of proinflammatory immune infiltrate relative to suppressive/exhausted infiltrate.

To directly demonstrate enhanced uptake of tumor tissue by tumor resident DC in DNP immune mice, but not DNP naïve mice, the mice are implanted with CFSE labeled tumor cells. The content of CSFE in the draining lymph node cross-presenting CD11c+CD103+CCR7+DC is determined by multiparameter flow cytometry. The role of the cross-presenting BATF3+DC is determined using BATF3 deficient mice.

Since VEGF or OPN receptors are also upregulated on the tumor endothelial cells, experiments are carried out to test whether an anti-endothelial immune response contributes to or is responsible for the observed anti-tumor response elicited by VEGF or OPN targeted coating with anti-DNP antibodies. Immunohistochemical analysis are done to determine if and to what extent the recruited antibodies and the VEGF or OPN-DNP conjugates bind to the tumor cells or the tumor endothelial cell (EC) in situ thus giving circumstantial indication which pathway could play a role. Preliminary evidence suggests that the VEGF targeted DNP conjugate bind predominantly to the tumor cells and not the tumor vasculature. To obtain functional evidence, it is hypothesized that whereas immunity to tumor expressed antigens will be tumor specific, immunity to normal endothelial cells will be cross-protective. To that end, the cross-protective nature of anti-tumor immune response is evaluated, for example, to assess whether the antibody coating mediated inhibition of 4T1 tumors induces protective immunity against CT26 tumors or vice versa. This is done by subsequent contralateral implantation of a second tumor, T cell transfer, or challenge of cured mice (the latter by using the immunogenic CT26 or A20 models as discussed elsewhere herein). If endothelial targeting is primarily responsible for the observed T cell dependent anti-tumor response, it will have important implications in term of the applicability of VEGF-targeted immune therapy to encompass virtually all cancer patients.

Alternative mechanism is assessed to determine the role of ADCC mediated killing of tumor vasculature endothelial cells. Given that tumor endothelial cells upregulate the receptors for VEGF and OPN, it is possible that the observed inhibition of tumor growth is mediated in a non T cell dependent manner by disruption of the tumor vasculature. This is achieved by coating the tumor endothelial cells with anti-DNP polyclonal antibodies in the VEGF or OPN-DNP treated tumor bearing mice, which are then killed and/or phagocytosed in an Fc-FcR mediated process by NK cells or macrophages/neutrophils, respectively. Evidence that would support such a mechanism may obtained from the experiments described elsewhere, (i) No effect of CD4 or CD8 cell depletion. (ii) Absence of an enhanced T cell mediated proinflammatory infiltrate. (iii) No enhanced uptake of tumor debris by DC (iv) Evidence of NK and or macrophage role. (v) Pattern of intratumoral distribution of IgG, IgM and VEGF- or OPN-DNP in terms of association with tumor cells and/or endothelial cells. Evidence showing that VEGF-DNP mediated tumor inhibition is CD4+ cell dependent argues against this mechanism.

In the DNP immune mice the VEGF/OPN-DNP conjugates administered into the circulation can bind the anti-DNP antibodies in the circulation, then migrate to the tumor and bind the VEGF or OPN receptors on the tumor cell or the endothelial cells. Alternatively, the VEGF/OPN-DNP conjugates can first traffic to the tumor, bind to the VEGF or OPN receptors, and then bind the anti-DNP antibodies. One reason that this may be important is that in the former case the large conjugate-antibody complex could be impeded from penetrating into the tumor and encounter the tumor cells. This will not matter if the tumor endothelial cells are the primary mediators of anti-tumor immunity. To determine if and to what extent the injected conjugates form complexes with the cognate antibodies in the circulation, blood sample are analyzed for the size of the injected VEGF-DNP conjugate over a period of up to 24 hours using size chromatography. Presence of DNP conjugates in eluted fractions is determined by ELISA using anti-DNP rabbit polyclonal antisera.

Evidence of toxicity in terms of morbidity or mortality in mice treated with aptamer-DNP conjugates has not been observed. The aptamer backbone contains 2′-fluoro-modified nucleotides which effectively eliminates their nonspecific immune activation properties. These properties were monitored by measuring TNF, IL-6 and type I IFN in the circulation, and no evidence was seen with multiple aptamers. Nonspecific inflammation is evaluated by counting CD4+ and CD8+ T cells in the liver, lymph nodes and spleen, and by H&E staining of liver, lung and intestines. Autoimmune pathology is assessed by measuring liver transaminases in the circulation, AST and ALT. Toxicity that could be associated with an antivasculature immune response is evaluated by measuring treatment effects on wound healing and pregnancy. Engagement of immune complexes by FcR-expressing phagocytes at far higher doses can induce a cytokine storm with pathological consequences known as “serum sickness,” which is monitored, or form deposits in various tissues (e.g., kidney) leading to glomerulonephritis that manifests as morbidity and possible mortality.

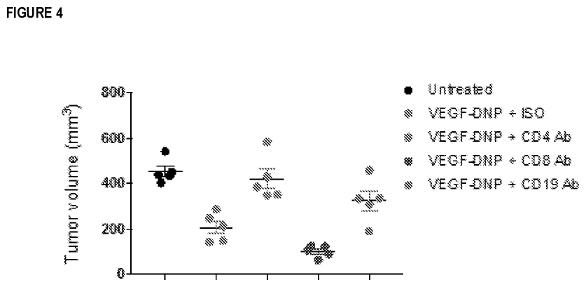

FIG. 4 shows immunological analysis involving antibody depletion experiments. Results indicated that the VEGF-DNP mediated anti-tumor response was mediated by CD4+, but not CD8+ T cells and dependent on a humoral response. This is consistent with a study showing that coating tumor cells with allogeneic antibody elicited primarily a CD4+, but not CD8+, T cell response. The incomplete abrogation of tumor inhibition by CD19 depletion was likely the reflection of only a partial reduction in circulating anti-DNP antibodies (˜75% at the time of tumor implantation).

Consistent with the postulated mechanism of action, immunoglobulin (Ig) deposits accumulated only in KLH-DNP, but not KLH, vaccinated mice treated systemically with VEGF-targeted DNP conjugates (FIG. 5). Notably, the Ig deposits associated mostly with the 4T1 tumor cells and not endothelial cells, despite the fact that endothelial cells in tumors, as well as the 4T1 tumor cells themselves, express VEGF receptors, suggesting that interactions of VEGF with heparan sulfate expressed on tumor cells dominated over interactions with its receptors.

### Example 4: Combination Immunotherapy

Combination therapy is carried out to evaluate whether judiciously chosen combination approaches can synergize with the antibody coating-enhanced tumor cell uptake approach. Since the antibody coating approach promotes the uptake of tumor-derived antigens by tumor-resident DC, it is believed that increasing the antigenic content of the tumor cells and/or the number of tumor-resident DC will enhance tumor inhibition.

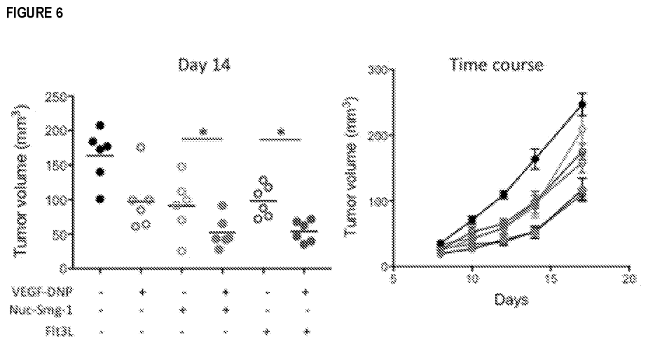

The antigenic content of tumor cells was increased using a unique approach to induce neoantigens in tumor lesion in situ by (aptamer) targeted inhibition of the Nonsense-mediated mRNA Decay (NMD) process that normally prevents the expression of defective products that otherwise would be seen by the immune system as neoantigens. Tumor targeted NMD inhibition was achieved by downregulating key mediators of the NMD pathways using corresponding siRNA that are targeted to the tumor by conjugation to an appropriate aptamer. In the experiments depicted in FIG. 6, a nucleolin-binding aptamer was used because nucleolin was translocated to the cell surface of most tumor cells of both murine and human origin. The nucleolin aptamer was conjugated to a siRNA that downregulated Smg-1, a key NMD factor. In a second combinatorial approach, mice were treated with Flt-3 ligand (Flt3L) to increase the numbers of tumor-resident. Batf3+CD103+ dendritic cells.

As shown in FIG. 6, using the palpable 4T1 model, both neoantigen induction in the tumor cells and Flt3L recruitment of DC when combined with subsequent VEGF-DNP mediated antibody coating of tumor cells enhanced tumor inhibition. Additional experiments directed to combination therapy are performed as described below.

Enhancing tumor antigen uptake by DC is one step in a multi-step process that has to occur substantially in order to engender effective protective anti-tumor immunity.

Improved antigen uptake with complementary immune potentiating strategies can significantly enhance anti-tumor immunity. In this regard, strategies that directly impact the antigen uptake process, namely increase the neoantigen content of the tumor cells, block the function of the inhibitory receptor CD47, attract DC to the tumor lesion are evaluated. Downstream immune potentiating strategies including checkpoint blockade and 4-1BB costimulation are also assessed. Combination therapies are screened in the transplantable 4T1 tumor model and selected combinations that exhibit superior anti-tumor activity are tested in the stringent and increasingly relevant BRAF mutant melanoma and PDA models.

Inducing Neoantigens in Tumor by Targeted Inhibition of Nonsense-Mediated mRNA (NMD)

Neoantigens are the most effective antigens to elicit protective anti-tumor immunity. Recent studies have demonstrated a strong correlation between the number of neoantigens expressed in tumor cells and responsiveness to checkpoint blockade therapy. These studies provided compelling clinical evidence that the tumor' neoantigen burden is a major determinant of tumor immunogenicity. Despite the patient-specific nature of most neoantigens, a broad effort in the academia and industry is currently focusing on developing clinically useful protocols to isolate neoantigens to be used in a vaccination protocol in combination with checkpoint blockade therapy, and for that matter other immune potentiating strategies.

Nonetheless, the majority of patients may not express sufficient neoantigens in their tumors to be responsive to immune therapy and recent studies have suggested that T cells recognizing such endogenous neoantigens are defective, perhaps irreversibly dysfunctional. Addressing these limitations, a simple, broadly applicable method of inducing neoantigens in tumor cells in situ was developed by inhibiting a process called Nonsense-mediated mRNA decay (NMD). This was achieved by downregulating key mediators of the NMD process using siRNAs that are targeted to the tumor lesions of mice by conjugation to an appropriate aptamer ligands. Aptamer-targeted siRNA inhibition of NMD in tumor cells in situ engendered potent anti-tumor immunity that was superior to that of conventional vaccination protocols.

A broadly useful targeting aptamer was utilized that bound to nucleolin, a nucleolar product that in most if not all tumor cells of both murine and human origin also translocates to the cell surface. Nucleolin aptamer targeted Smg-1 siRNA delivery (Smg-1 is a key mediator of NMD) was effective in controlling tumor growth in multiple tumor models including 4T1, A20, CT26. Additional methods were tested of inducing neoantigens tumor lesions by targeted downregulation of key mediators of antigen processing pathways like TAP, ERAAP or Invariant chain. Without wishing to be bound by theory, it is believed that improved tumor uptake by the DC can be enhanced by first expressing neoantigens in the tumor.

### Enhancing the Intratumoral Accumulation of Proinflammatory Immune Cells.

Tumor lesions are poorly infiltrated by immune cells which is a main reason why they are not responsive to checkpoint blockade therapy, and conceivably other forms of immune potentiating therapies. Further, neoantigen burden itself does not appear to be sufficient to promote immune infiltration. It was recently shown that one mechanism preventing the intratumoral trafficking of immune cells is mediated by the wnt/β-catenin pathway, and that absence of β-catenin expression in tumor cells converts “noninflamed” into “inflamed” tumors. 4T1 breast carcinoma and B16.F10 melanoma cells express elevated levels of β-catenin and its downstream mediator TCF7, i.e., at 5-8 fold higher level than syngeneic adherent splenocytes or contact inhibited NIH 3T3 cells as determined by qRT-PCR. It is believed that tumor targeted downregulation of β-catenin in 4T1 tumors in situ using nucleolin or EpCAM aptamer-siRNA conjugates enhances intratumoral T cell infiltration and synergize with the antibody coating approach.

Other methods to promote intratumoral immune infiltration that may be utilized include local irradiation or intratumoral administration of STING ligand.

### Increasing the DC Content of Tumors by Flt-3 Ligand (Flt3L) Administration

Naturally growing tumors may be poorly infiltrated by DC. Thus increasing their numbers will improve the outcome of immune therapy. In the tumor microenvironment, the antigen presenting DC responsible for stimulating anti-tumor T cell immunity are a rare population of CD103+Batf3+IRF8+DC (CD141+DC in humans). The CD103+DC that also express CCR7, acquire tumor antigen at the tumor site, migrate to the draining lymph node where they activate cognate T cells either directly or by handing-off to resident DC. One way to increase the overall and tumor resident DC content is systemic administration of Flt3L, a cytokine that promotes hematopoietic progenitor commitment to the DC lineage as well as the survival and proliferation of DC in tissues. Administration of Flt3L to tumor bearing mice enhanced intratumoral accumulation of DC and tumor inhibition. It is believed that Flt3L administration to the tumor bearing mice will synergize with subsequent treatment with the aptamer-DNP conjugate, leading to increased DC content around tumor cells and enhanced anti-tumor immunity.

### CD47 Blockade

CD47 is an inhibitory receptor that prevents phagocytic uptake of CD47 expressing cells. CD47 is upregulated on many tumors, and treatment of tumor bearing mice with blocking anti-CD47 antibodies inhibited tumor growth in multiple models. CD47 antibody therapy is now in clinical testing in patients with AML. CD47 can be blocked using a commercially available depleting antibody from BioXcell. It is believed that CD47 blockage will synergize with treatment with the aptamer-DNP conjugate in treating tumor.

### Enhancing the Survival and Proliferative Capacity of Tumor Infiltrating T Cells

It is believed that enhancing the survival and proliferative capacity of tumor infiltrating T cells may also synergize with treatment with the aptamer-DNP conjugate in tumor inhibition. Specifically, the survival and proliferative capacity of tumor infiltrating T cells may be achieved by blocking inhibitory receptors and engaging stimulatory receptors.

CTLA-4 and PD-1 antibodies are used to counter the function of inhibitory receptors expressed on tumor infiltrating T cells. Yet despite unprecedented clinical responses as monotherapy, use of these antibodies is not a cure, and a significant fraction of patients do not respond and/or in the case of CTLA-4 therapy can exhibit significant toxicity.

Experiments are carried to test if co-therapy with CTLA-4 and/or PD-1 antibody will synergize with VEGF-DNP mediated enhanced antigen uptake. Particular attention is given to monitoring toxicities in mice treated with CTLA-4 antibodies. Enterocolitis and inflammation of the intestine are the main severe toxicity seen in patients treated with CTLA-4 antibody (Ipilumimab). The toxicities seen with CTLA-4 antibodies were recapitulated in mice, both as monotherapy or in combination with tumor radiation, characterized by significant inflammatory responses in the intestine, lung and liver, with histological evidence of tissue damage in the intestine.

Alternatively or in combination, 4-1BB costimulation is promoted on the tumor infiltrating T cells. 4-1BB is a major immune stimulatory receptor expressed on activated CD8+ T cells including tumor infiltrating CD8+ T cells. Engagement by its ligand promotes the survival and proliferative capacity of (vaccine/tumor) antigen activated CD8+ T cells. Systemic administration of agonistic 4-1BB antibodies to mice potentiates anti-tumor immunity, but also elicits organ wide inflammatory responses and liver damage, and severe liver toxicity in cancer patients.

An approach was developed to overcome the toxicity associated with 4-1BB therapy by targeting 4-1BB costimulation to the tumor lesion. Briefly, the approach was to use a 4-1BB binding aptamer that acts as the agonistic ligand that was targeted to the tumor by conjugation to VEGF or OPN binding aptamer ligands. Tumor targeted 4-1BB costimulation appeared to be exceptionally effective as judged by using increasingly relevant and stringent murine tumor models. Unlike nontargeted 4-1BB antibody administration, the aptamer targeted approach was devoid of measurable autoimmune pathologies. It is believed that tumor targeted 4-1BB costimulation enhances the anti-tumor responses of the aptamer-DNP mediated enhanced tumor antigen uptake. Further, the combination therapy, in contrast to treatment with 4-1BB or CTLA-4 antibodies alone, does not induce significant toxicities.

Additional immunotherapies including Treg elimination, IDO inhibition, LIGHT, OX40, GITR stimulation, STING ligand IFNα induction may also synergize with aptamer-DNP conjugates in tumor treatment.

### Example 5: Methods to Coat Tumor Cells In Situ with Anti-αGal Natural Antibodies

Natural antibodies against the trisaccharide epitope Galα1-3Galβ1-4GlcNAc-R (αGal) are the most abundant antibody (1% of total immunoglobulin) in the sera of human. They are produced throughout life as a result of constant antigenic stimulation by carbohydrate antigens on commensal bacteria in the GI tract. Thus “coating” tumor cells with the αGal epitope may dispense with the need to vaccinate. Wild type mice lack the ability to produce anti-αGal antibodies, since it is synthesized in mice and therefore it is a self-antigen to which mice are tolerant to. ThaII and colleagues have developed an αGal deficient mouse strain by deleting the α1,3 galactose transferase gene (GT KO mice). While αGal antibody titers in the GT KO mice housed in vivarium with reduced exposure to commensal bacteria is low, immunization with xenograft tissue such as rabbit red blood cells or pig kidney membrane homogenate, induced high levels of anti-αGal antibodies comparable to that found in human serum. Thus the αGal vaccinated GT KO mice may be utilized as a model for developing strategies that exploit the natural anti-αGal response for the currently described studies.

Unlike human tumors that are αGal null, the GT+ mouse tumors express αGal glycoproteins that would be readily rejected in the GT KO mice immunized against αGal with the pig homogenates. αGal null tumor cell lines are generated by lentiviral expression of a GT shRNA. Alternatively, B6BL6 tumors, a highly metastatic subline of B16 tumors which has downregulated GT and does not express αGal, may be used as a model for human αGal null tumors.

### Generation and Characterization of VEGF and OPN-αgal Conjugates

An ODN-αGal fusion for hybridization to aptamer or scaffold was generated. For the study, the monovalent and multivalent conjugates are tested for binding to VEGF, and OPN receptor expressing cells are stained in vitro with a commercially available anti-αGal monoclonal antibody (M86, ENZO), with human sera, and αgal antibody depleted control human sera (passing thru a column immobilized with αGal-ODN), as well as with sera from GT-deficient mice vaccinated or not against αGal.

Inhibition of Tumor Growth in Mice Treated with VEGF and OPN-αGal Conjugates

A GT deficient colony (GT KO) was established. The mice were of mixed background (C57BL/6xDBA/2Jx129sv GT−/−) because homozygous GT KO H-2b mice do not breed well and produce reduced anti-αGal titers. B6BL6 melanoma tumors of H-2b background can grow in these mice by injecting ˜5×10⁵ cells. This was consistent with genotyping 4 males and 4 female mice picked at random from the colony that contained 45%-60% contribution from the H-2b genotype. The low titers of αGal antibodies are boosted by immunization with pig kidney membrane homogenates obtained from a commercial vendor (Pelfreeze). Immunized and nonimmunized mice are implanted subcutaneously with B6BL6 tumor cells and treated with the best-in-class in vitro characterized VEGF and OPN-αGal conjugates. Combination therapies are tested along with immunological and toxicity studies.

### Example 6: Enhancing the ADCC Functions of Therapeutic Antibodies

Clinical efficacy of antitumor antibodies targeted to cell surface products expressed on tumor cells such CD20 (Rituximab), Her2 (Trastuzamab), or EGFR (Cetuximab) is mediated at least in part via Fc-FcR mediated ADCC. Specifically, the dramatic effects of checkpoint blockade antibodies against CTLA-4 or PD-L1 may be mediated primarily via Fc-FcR depletion of intratumoral Treg and F4/80+ myeloid cells, respectively. A broad effort in the industry and academia is currently focused on improving the ADCC function of therapeutic antibodies by genetic engineering of their Fc portion. Yet given the noted synergy between antibodies of complementary mode of action encoded in their Fc portions, short of judicious combination of two or more monoclonal antibodies, their monofunctionality could potentially limit their therapeutic efficacy.

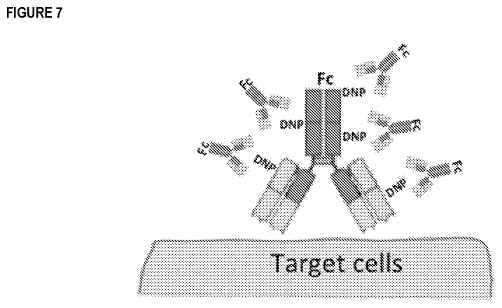

The present invention relates to an alternative method to improve the ADCC function of therapeutic antibodies which is a much simpler and straightforward alternative to genetically engineering one antibody at a time. The present method is also potentially more effective by virtue of engaging polyclonal antibodies. By way of example, as depicted in FIG. 7, the method involves “decorating” the therapeutic monoclonal antibody, e.g., Rituximab, Cetuximab, with haptens like DNP that are administered to patients pre-immunized against DNP. Here the monoclonal antibody fulfills a targeting, not therapeutic, function to decorate the target (tumor) cells with DNP hapten which in turn will attract the pre-formed anti-DNP antibodies, in effect “coating” the target cells with polyclonal antibodies.

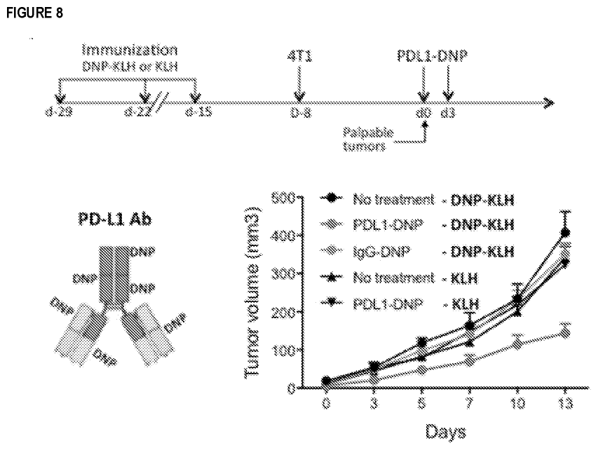

FIG. 8 shows that the therapeutic efficacy of an anti-PD-L1 antibody may be improved by methods of the invention. Treatment of cancer patients with monoclonal anti-PD-L1 antibodies as monotherapy exhibits a modest though significant therapeutic benefit. It has been assumed that the PD-L1 antibodies function via blocking PD-1/PD-L1 interactions, i.e., checkpoint blockade. However, a recent study in mice has shown that the antitumor effect of PD-L1 antibody could also be mediated via ADCC of PD-L1 expressing tumor infiltrating immune suppressive F4/80 macrophages.

Experiments were carried out to demonstrate that a DNP “decorated’ PD-L1 antibody elicited a more potent antitumor immune response in mice pre-vaccinated against DNP. As shown in FIG. 8, a murine PD-L1 monoclonal antibody was first decorated with multiple copies of DNP, a modification that did not affect the binding ability or in vivo antitumor activity of the DNP modified PD-L1 antibody. Specifically, the murine PD-L1 antibody was modified with 5-8 DNP moieties by chemical conjugation of an NHS modified DNP to the primary amine groups of lysines. Mice were immunized against DNP with DNP-KLH or mock immunized with KLH, and subsequently implanted subcutaneously with 4T1 breast carcinoma tumor cells. About 7-8 days later when tumors became palpable, mice were treated with the PD-L1-DNP antibody by systemic administration via tail vein injections. Dose was adjusted to 100 μg/injection so that PD-L1 Ab as monotherapy had a minimal to no effect on tumor growth. As shown in FIG. 8, a significant inhibition of tumor growth was noted when the DNP-modified PD-L1 Ab was injected into mice pre-vaccinated against DNP, but not control mice vaccinated against KLH.

These results strong indicate modifying monoclonal antibodies with haptens and treating patients pre-vaccinated against the hapten significantly enhanced their FcR-mediated functions and their antitumor activity. In alternative embodiments, haptens recognized by naturally occurring antibodies may be utilized thereby dispensing with the need for vaccination.

### Example 7: Dispensing with Vaccination by Recruiting Naturally Occurring Antibodies

The protocol to coat tumor cells in situ with endogenous polyclonal antibodies as depicted in FIG. 1 consists of two steps, vaccination against an antigen/hapten and treatment with a tumor-targeted said antigen/hapten. While the vaccination step is simple and carries low risk, it is possible to dispense with vaccination altogether by recruiting naturally occurring antibodies. Natural antibodies against the trisaccharide epitope Galα1-3Galβ1-4GlcNAc-R (αGal) are the most abundant antibody (1% of total immunoglobulin) in the sera of humans, produced throughout life as a result of constant antigenic stimulation by carbohydrate antigens on commensal bacteria in the GI tract. Wild type mice do not produce anti-αGal antibody, since it is synthesized in mice and therefore it is a self-antigen to which mice are tolerant.

To model the recruitment of natural antibodies in mice, an αGal deficient mouse strain that was generated by deleting the α1,3 galactose transferase, a key enzyme in the synthesis of αGal (GT KO mice) was used. While □Gal antibody titers in the GT KO mice housed in the vivarium with reduced exposure to commensal bacteria is low, immunization with xenograft tissue such as rabbit red blood cells or pig kidney membrane homogenate, induces high levels of anti-αGal antibodies comparable to that found in human serum. Unlike human tumors that are αGal null, mouse tumors express αGal containing glycoproteins and thereby are rejected in the GT KO mice immunized against αGal. Since B16BL6 tumors, a highly metastatic subline of B16 tumors, has downregulated GT and does not express αGal, it was used as a suitable model for human αGal null tumors.

The GT KO mice are of mixed genetic background (C57BU6xDBA/2Jx129sv GT−/−) because homozygous GT KO H-2b mice don't breed well and produce reduced anti-αGal titers. In the colony established, the contribution of H-2b genotype varied from 40-60%. The low titers of αGal antibodies in the GT KO mice were boosted by immunization with pig kidney membrane homogenates.

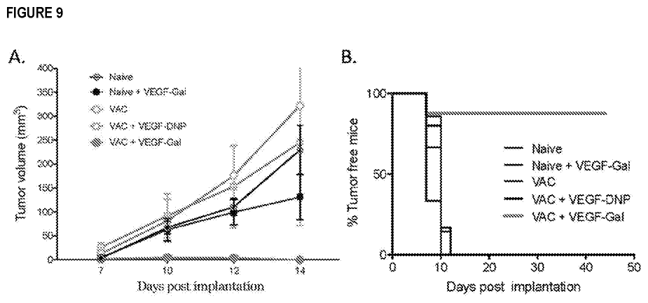

To test whether tumor recruitment of the αGal antibodies could inhibit tumor growth, mice implanted with B16BL6 tumors were treated with VEGF-targeted αGal trisaccharide. B16BL6 tumor cells, derived from the metastatic B16.F10 melanoma tumor cell line, grow in the GT KO mice and injecting ˜5×10⁵ cells. Briefly, αGal-null (C57BL/6xDBA/2Jx129sv GT−/−) mice were vaccinated with pig kidney extracts to boost the titer of anti-αGal antibodies and 10 day later challenged subcutaneously with 5×10⁵ αGal^{low/negative} B16BL6 tumor cells. 3 days' post tumor implantation mice were treated with VEGF-DNP or VEGF-αGal hapten conjugates and tumor growth was measured. As shown in FIGS. 9A-B, tumors failed to grow in the VEGF-αGal treated mice, 5/6 mice remaining tumor free for the duration of the experiment, provided the mice were boosted with the kidney membrane homogenates. Importantly, vaccinated mice treated with VEGF-DNP conjugates did not reject the B16BL6 tumors. This experiment, therefore, shows that tumor rejection was dependent on the endogenous αGal antibodies and their recruitment to the tumor. Indicative of establishment of immunological memory, tumors failed to grow in 4 out of 5 mice that rejected the initial tumor challenge whereas they grew in 7 out of 7 non vaccinated GT KO mice.

### Example 8: Potential Relevance to Human Patients

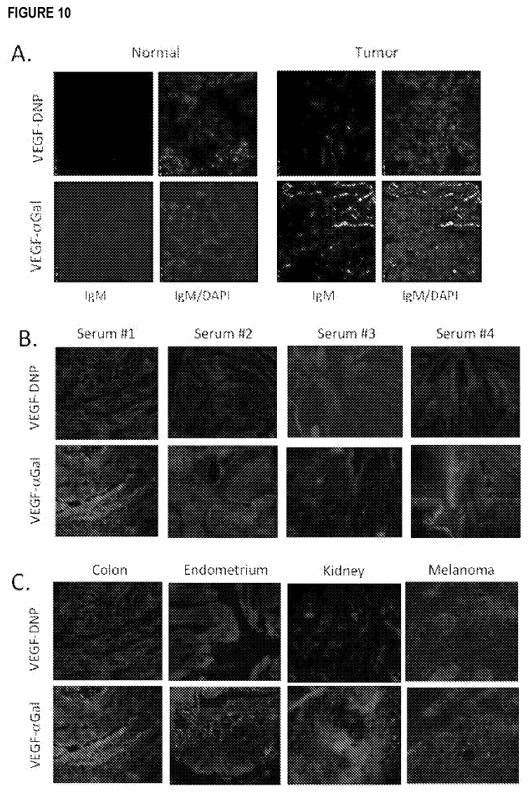

To assess the potential relevance to human patients, it was examined whether VEGF aptamer targeted αgal hapten could recruit antibodies present in the human serum to tumors, but not to normal tissue. Frozen sections of tumor and matched normal tissue from the same patient were first incubated with VEGF aptamer-αgal conjugate or with VEGF aptamer-DNP conjugate used as a control, and then incubated with human serum and stained with either DAPI (blue) or with mouse anti-human IgM-Alexa488 A (green) and visualized by confocal microscopy at 40× magnification. A. Renal cell cancer (RCC) and matched normal tissue. As shown in a representative example in FIG. 10A, human serum bound to a tumor section obtained from a kidney tumor biopsy, but not to normal tissue from the same individual, that was incubated with VEGF-αgal but not with VEGF-DNP conjugates. FIG. 10B shows that four sera obtained from healthy donors recruited antibodies to tumor, consistent with the prevalence of anti-αgal antibodies in the human population. FIG. 100 shows that VEGF-αgal, but not VEGF-DNP, conjugate recruited antibodies to tumors of endometrial, kidney, melanoma, and colon origin, altogether 5 out of 5 tumors tested, reflecting the broad expression pattern of VEGF in tumors and the utility of VEGF aptamer targeting ligand, as we have previously demonstrated in mice. The variation in staining intensities seen in FIG. 10B and FIG. 10C may reflect variation in the titer of anti-αGal antibodies or intratumoral VEGF expression, respectively. Taken together, the experiments shown in FIGS. 10 A-C, support the view that treatment with VEGF aptamer targeted αgal conjugate will be capable of recruiting polyclonal antibodies to the tumors of most if not all cancer patients.

In summary, most tumors can be targeted via VEGF which is expressed in many tumors or can be upregulated by irradiation, as illustrated (FIG. 10C). Whereas vaccination against a hapten or antigen is a simple and low risk procedure it could be dispensed altogether by recruiting naturally occurring antibodies like the highly abundant αGal-specific antibodies that constitute about 1% of the total circulating immunoglobulin in the human serum. Using a murine model that simulates the scenario in human patients we have shown that treatment of the tumor bearing mice with VEGF aptamer targeted αGal hapten prevented tumor growth (Figure. 9), and recruited antibodies from 4/4 human sera tested (FIG. 10B) to 5/5 human tumor sections of distinct origin (FIG. 10C). Taken together, these experiments suggest that treatment with a systemically administered, chemically synthesized, broadly applicable drug could recruit preexisting antibodies and sensitize any tumor lesion in every cancer patient to the immune system and thereby contribute to control of tumor growth, especially when used in combination with other immune potentiating treatments.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

1. Chao M P, Weissman I L, and Majeti R. The CD47-SIRPalpha pathway in cancer immune evasion and potential therapeutic implications. Curr Opin Immunol. 2012; 24(2):225-32.
2. Willingham S B, Volkmer J P, Gentles A J, Sahoo D, Dalerba P, Mitra S S, Wang J, Contreras-Trujillo H, Martin R, Cohen J D, et al. The CD47-signal regulatory protein alpha (SIRPa) interaction is a therapeutic target for human solid tumors. Proc Natl Acad Sci USA. 2012; 109(17):6662-7.
3. DiLillo D J, and Ravetch J V. Fc-Receptor Interactions Regulate Both Cytotoxic and Immunomodulatory Therapeutic Antibody Effector Functions. Cancer immunology research. 2015; 3(7):704-13.
4. Carmi Y, Spitzer M H, Linde I L, Burt B M, Prestwood T R, Perlman N, Davidson M G, Kenkel J A, Segal E, Pusapati G V, et al. Allogeneic IgG combined with dendritic cell stimuli induce antitumour T-cell immunity. Nature. 2015; 521(7550):99-104.
5. Majumder K, Arora N, Modi S, Chugh R, Nomura A, Giri B, Dawra R, Ramakrishnan S, Banerjee S, Saluja A, et al. A Novel Immunocompetent Mouse Model of Pancreatic Cancer with Robust Stroma: a Valuable Tool for Preclinical Evaluation of New Therapies. Journal of gastrointestinal surgery: official journal of the Society for Surgery of the Alimentary Tract. 2016; 20(1):53-65; discussion
6. Postow M A, Callahan M K, and Wolchok J D. Immune Checkpoint Blockade in Cancer Therapy. J Clin Oncol. 2015; 33(17):1974-82.
7. Chao M P, Majeti R, and Weissman I L. Programmed cell removal: a new obstacle in the road to developing cancer. Nat Rev Cancer. 2012; 12(1):58-67.
8. Majeti R, Chao M P, Alizadeh A A, Pang W W, Jaiswal S, Gibbs K D, Jr., van Rooijen N, and Weissman I L. CD47 is an adverse prognostic factor and therapeutic antibody target on human acute myeloid leukemia stem cells. Cell. 2009; 138(2):286-99.
9. Jaiswal S, Jamieson C H, Pang W W, Park C Y, Chao M P, Majeti R, Traver D, van Rooijen N, and Weissman I L. CD47 is upregulated on circulating hematopoietic stem cells and leukemia cells to avoid phagocytosis. Cell. 2009; 138(2):271-85.
10. Liu X, Pu Y, Cron K, Deng L, Kline J, Frazier W A, Xu H, Peng H, Fu Y X, and Xu M M. CD47 blockade triggers T cell-mediated destruction of immunogenic tumors. Nat Med. 2015; 21(10):1209-15.
11. Bournazos S, and Ravetch J V. Fcgamma receptor pathways during active and passive immunization. Immunol Rev. 2015; 268(1):88-103.
12. Bruhns P, and Jonsson F. Mouse and human FcR effector functions. Immunol Rev. 2015; 268(1):25-51.
13. Uchida J, Hamaguchi Y, Oliver J A, Ravetch J V, Poe J C, Haas K M, and Tedder T F. The innate mononuclear phagocyte network depletes B lymphocytes through Fc receptor-dependent mechanisms during anti-CD20 antibody immunotherapy. J Exp Med. 2004; 199(12):1659-69.
14. Musolino A, Naldi N, Bortesi B, Pezzuolo D, Capelletti M, Missale G, Laccabue D, Zerbini A, Camisa R, Bisagni G, et al. Immunoglobulin G fragment C receptor polymorphisms and clinical efficacy of trastuzumab-based therapy in patients with HER-2/neu-positive metastatic breast cancer. J Clin Oncol. 2008; 26(11):1789-96.
15. Bibeau F, Lopez-Crapez E, Di Fiore F, Thezenas S, Ychou M, Blanchard F, Lamy A, Penault-Llorca F, Frebourg T, Michel P, et al. Impact of Fc{gamma}RIIa-Fc{gamma}RIIIa polymorphisms and KRAS mutations on the clinical outcome of patients with metastatic colorectal cancer treated with cetuximab plus irinotecan. J Clin Oncol. 2009; 27(7):1122-9.
16. Selby M J, Engelhardt J J, Quigley M, Henning K A, Chen T, Srinivasan M, and Korman A J. Anti-CTLA-4 antibodies of IgG2a isotype enhance anti-tumor activity through reduction of intratumoral regulatory T cells. Cancer immunology research. 2013; 1(1):32-42.
17. Simpson T R, Li F, Montalvo-Ortiz W, Sepulveda M A, Bergerhoff K, Arce F, Roddie C, Henry J Y, Yagita H, Wolchok J D, et al. Fc-dependent depletion of tumor-infiltrating regulatory T cells co-defines the efficacy of anti-CTLA-4 therapy against melanoma. J Exp Med. 2013; 210(9):1695-710.
18. Bulliard Y, Jolicoeur R, Windman M, Rue S M, Ettenberg S, Knee D A, Wilson N S, Dranoff G, and Brogdon J L. Activating Fc gamma receptors contribute to the anti-tumor activities of immunoregulatory receptor-targeting antibodies. J Exp Med. 2013; 210(9):1685-93.
19. Dahan R, Sega E, Engelhardt J, Selby M, Korman A J, and Ravetch J V. FcgammaRs Modulate the Anti-tumor Activity of Antibodies Targeting the PD-1/PD-L1 Axis. Cancer Cell. 2015; 28(3):285-95.
20. Li F, and Ravetch J V. A general requirement for FcgammaRIIB co-engagement of agonistic anti-TNFR antibodies. Cell Cycle. 2012; 11(18):3343-4.
21. Rafiq K, Bergtold A, and Clynes R. Immune complex-mediated antigen presentation induces tumor immunity. J Clin Invest 2002; 110(1):71-9.
22. Vasovic L V, Dyall R, Clynes R A, Ravetch J V, and Nikolic-Zugic J. Synergy between an antibody and CD8+ cells in eliminating an established tumor. Eur J Immunol. 1997; 27(2):374-82.
23. de Bono J S, Rha S Y, Stephenson J, Schultes B C, Monroe P, Eckhardt G S, Hammond L A, Whiteside T L, Nicodemus C F, Cermak J M, et al. Phase I trial of a murine antibody to MUC1 in patients with metastatic cancer: evidence for the activation of humoral and cellular anti-tumor immunity. Annals of oncology official journal of the European Society for Medical Oncology/ESMO. 2004; 15(12):1825-33.
24. Hilchey S P, Hyrien O, Mosmann T R, Livingstone A M, Friedberg J W, Young F, Fisher R I, Kelleher R J, Jr., Bankert R B, and Bernstein S H. Rituximab immunotherapy results in the induction of a lymphoma idiotype-specific T-cell response in patients with follicular lymphoma: support for a “vaccinal effect” of rituximab. Blood. 2009; 113(16):3809-12.
25. Kwak L W, Campbell M J, Czerwinski D K, Hart S, Miller R A, and Levy R. Induction of immune responses in patients with B-cell lymphoma against the surface-immunoglobulin idiotype expressed by their tumors. N Engl J Med. 1992; 327(17):1209-15.
26. Taylor C, Hershman D, Shah N, Suciu-Foca N, Petrylak D P, Taub R, Vandat L, Cheng B, Pegram M, Knutson K L, et al. Augmented HER-2 specific immunity during treatment with trastuzumab and chemotherapy. Clin Cancer Res. 2007; 13(17):5133-43.
27. Broz M L, Binnewies M, Boldajipour B, Nelson A E, Pollack J L, Erle D J, Barczak A, Rosenblum M D, Daud A, Barber D L, et al. Dissecting the tumor myeloid compartment reveals rare activating antigen-presenting cells critical for T cell immunity. Cancer Cell. 2014; 26(5):638-52.
28. Roberts E W, Broz M L, Binnewies M, Headley M B, Nelson A E, Wolf D M, Kaisho T, Bogunovic D, Bhardwaj N, and Krummel M F. Critical Role for CD103(+)/CD141(+) Dendritic Cells Bearing CCR7 for Tumor Antigen Trafficking and Priming of T Cell Immunity in Melanoma. Cancer Cell. 2016; 30(2):324-36.
29. Dhodapkar K M, Kaufman J L, Ehlers M, Banerjee D K, Bonvini E, Koenig S, Steinman R M, Ravetch J V, and Dhodapkar M V. Selective blockade of inhibitory Fcgamma receptor enables human dendritic cell maturation with IL-12p70 production and immunity to antibody-coated tumor cells. Proc Natl Acad Sci U S A. 2005; 102(8):2910-5.
30. Kalergis A M, and Ravetch J V. Inducing tumor immunity through the selective engagement of activating Fcgamma receptors on dendritic cells. J Exp Med. 2002; 195(12):1653-9.
31. Shinkawa T, Nakamura K, Yamane N, Shoji-Hosaka E, Kanda Y, Sakurada M, Uchida K, Anazawa H, Satoh M, Yamasaki M, et al. The absence of fucose but not the presence of galactose or bisecting N-acetylglucosamine of human IgG1 complex-type oligosaccharides shows the critical role of enhancing antibody-dependent cellular cytotoxicity. J Biol Chem. 2003; 278(5):3466-73.
32. DiLillo D J, and Ravetch J V. Differential Fc-Receptor Engagement Drives an Anti-tumor Vaccinal Effect. Cell. 2015; 161(5):1035-45.
33. Smith P, DiLillo D J, Bournazos S, Li F, and Ravetch J V. Mouse model recapitulating human Fcgamma receptor structural and functional diversity. Proc Natl Acad Sci USA. 2012; 109(16):6181-6.
34. Brandsma A M, Ten Broeke T, Nederend M, Meulenbroek L A, van Tetering G, Meyer S, Jansen J H, Beltran Buitrago M A, Nagelkerke S Q, Nemeth I, et al. Simultaneous Targeting of FcgammaRs and FcalphaRI Enhances Tumor Cell Killing. Cancer immunology research. 2015; 3(12):1316-24.
35. Reali E, Greiner J W, Corti A, Gould H J, Bottazzoli F, Paganelli G, Schlom J, and Siccardi A G. IgEs targeted on tumor cells: therapeutic activity and potential in the design of tumor vaccines. Cancer Res. 2001; 61(14):5517-22.
36. Nigro E A, Brini A T, Soprana E, Ambrosi A, Dombrowicz D, Siccardi A G, and Vangelista L. Anti-tumor IgE adjuvanticity: key role of Fc epsilon R I. J Immunol. 2009; 183(7):4530-6.
37. Nigro E A, Brini A T, Yenagi V A, Ferreira L M, Achatz-Straussberger G, Ambrosi A, Sanvito F, Soprana E, van Anken E, Achatz G, et al. Cutting Edge: IgE Plays an Active Role in Tumor Immunosurveillance in Mice. J Immunol. 2016; 197(7):2583-8.
38. Ott M G, Marme F, Moldenhauer G, Lindhofer H, Hennig M, Spannagl R, Essing M M, Linke R, and Seimetz D. Humoral response to catumaxomab correlates with clinical outcome: results of the pivotal phase II/III study in patients with malignant ascites. Int J Cancer. 2012; 130(9):2195-203.
39. Frank M M, and Hester C G. Immune Complexes: Normal Physiology and Role in Disease. Springer; 2009.
40. Murelli R P, Zhang A X, Michel J, Jorgensen W L, and Spiegel D A. Chemical control over immune recognition: a class of antibody-recruiting small molecules that target prostate cancer. Journal of the American Chemical Society. 2009; 131(47):17090-2.
41. Dubrovska A, Kim C, Elliott J, Shen W, Kuo T H, Koo D I, Li C, Tuntland T, Chang J, Groessl T, et al. A chemically induced vaccine strategy for prostate cancer. ACS chemical biology. 2011; 6(11):1223-31.
42. Kristian S A, Hwang J H, Hall B, Leire E, Iacomini J, Old R, Galili U, Roberts C, Mullis K B, Westby M, et al. Retargeting pre-existing human antibodies to a bacterial pathogen with an alpha-Gal conjugated aptamer. J Mol Med (Berl). 2015; 93(6):619-31.
43. Berezhnoy A, Castro I, Levay A, Malek T R, and Gilboa E. Aptamer-targeted inhibition of mTOR in T cell enhances anti-tumor immunity. J Clin Invest. 2014; 124(188-97.
44. Pastor F, Kolonias D, Giangrande P H, and Gilboa E. Induction of tumor immunity by targeted inhibition of nonsense mediated mRNA decay. Nature. 2010; 465(227-31.
45. Pastor F, Kolonias D, McNamara J O, 2nd, and Gilboa E. Targeting 4-1BB costimulation to disseminated tumor lesions with bi-specific oligonucleotide aptamers. Mol Ther. 2011; 19(10):1878-86.
46. Dassie J P, and Giangrande P H. Current progress on aptamer-targeted oligonucleotide therapeutics. Therapeutic delivery. 2013; 4(12):1527-46.
47. Zhou J, and Rossi J J. Cell-type-specific, Aptamer-functionalized Agents for Targeted Disease Therapy. Molecular therapy Nucleic acids. 2014; 3(e169.
48. Gilboa E, Berezhnoy A, and Schrand B. Reducing Toxicity of Immune Therapy Using Aptamer-Targeted Drug Delivery. Cancer immunology research. 2015; 3(11):1195-200.
49. Dvorak H F. Vascular permeability factor/vascular endothelial growth factor: a critical cytokine in tumor angiogenesis and a potential target for diagnosis and therapy. J Clin Oncol. 2002; 20(21):4368-80.
50. Senger D R, Van de Water L, Brown L F, Nagy J A, Yeo K T, Yeo T K, Berse B, Jackman R W, Dvorak A M, and Dvorak H F. Vascular permeability factor (VPF, VEGF) in tumor biology. Cancer metastasis reviews. 1993; 12(3-4):303-24.
51. Schrand B, Berezhnoy A, Brenneman R, Williams A, Levay A, Kong L Y, Rao G, Zhou S, Heimberger A B, and Gilboa E. Targeting 4-1BB costimulation to the tumor stroma with bispecific aptamer conjugates enhances the therapeutic index of tumor immunotherapy. Cancer immunology research. 2014; 2(9):867-77.
52. Schrand B, Verma B, Levay A, Patel S, Castro I, Benaduce A P, Brenneman R, Umland O, Yagita H, Gilboa E, et al. Radiation-induced VEGF-targeted 4-1BB costimulation enhances immune control of tumor growth. Cancer Res. 2017; In press
53. Wai P Y, and Kuo P C. The role of Osteopontin in tumor metastasis. The Journal of surgical research. 2004; 121(2):228-41.
54. Ferrara N, Gerber H P, and LeCouter J. The biology of VEGF and its receptors. Nat Med. 2003; 9(6):669-76.
55. Ellis L M, and Hicklin D J. VEGF-targeted therapy: mechanisms of anti-tumour activity. Nat Rev Cancer. 2008; 8(8):579-91.
56. Cohen T, Gitay-Goren H, Sharon R, Shibuya M, Halaban R, Levi B Z, and Neufeld G. VEGF121, a vascular endothelial growth factor (VEGF) isoform lacking heparin binding ability, requires cell-surface heparan sulfates for efficient binding to the VEGF receptors of human melanoma cells. J Biol Chem. 1995; 270(19):11322-6.
57. Houck K A, Leung D W, Rowland A M, Winer J, and Ferrara N. Dual regulation of vascular endothelial growth factor bioavailability by genetic and proteolytic mechanisms. J Biol Chem. 1992; 267(36):26031-7.
58. Darland D C, Massingham L J, Smith S R, Piek E, Saint-Geniez M, and D'Amore P A. Pericyte production of cell-associated VEGF is differentiation-dependent and is associated with endothelial survival. Dev Biol. 2003; 264(1):275-88.
59. Salvador C, Li B, Hansen R, Cramer D E, Kong M, and Yan J. Yeast-derived beta-glucan augments the therapeutic efficacy mediated by anti-vascular endothelial growth factor monoclonal antibody in human carcinoma xenograft models. Clin Cancer Res. 2008; 14(4):1239-47.
60. Cressey R, Wattananupong O, Lertprasertsuke N, and Vinitketkumnuen U. Alteration of protein expression pattern of vascular endothelial growth factor (VEGF) from soluble to cell-associated isoform during tumourigenesis. BMC cancer. 2005; 5(128.
61. Wei Y Q, Wang Q R, Zhao X, Yang L, Tian L, Lu Y, Kang B, Lu C J, Huang M J, Lou Y Y, et al. Immunotherapy of tumors with xenogeneic endothelial cells as a vaccine. Nat Med. 2000; 6(10):1160-6.
62. Wagner S C, Riordan N H, Ichim T E, Szymanski J, Ma H, Perez J A, Lopez J, Plata-Munoz J J, Silva F, Patel A N, et al. Safety of targeting tumor endothelial cell antigens. J Trans' Med. 2016; 14(90.
63. Matejuk A, Leng Q, Chou S T, and Mixson A J. Vaccines targeting the neovasculature of tumors. Vascular cell. 2011; 3(1):7.
64. Li Y, Wang M N, Li H, King K D, Bassi R, Sun H, Santiago A, Hooper A T, Bohlen P, and Hicklin D J. Active immunization against the vascular endothelial growth factor receptor flk1 inhibits tumor angiogenesis and metastasis. J Exp Med. 2002; 195(12):1575-84.
65. Luo Y, Zhou H, Krueger J, Kaplan C, Lee S H, Dolman C, Markowitz D, Wu W, Liu C, Reisfeld R A, et al. Targeting tumor-associated macrophages as a novel strategy against breast cancer. J Clin Invest. 2006; 116(8):2132-41.
66. Nair S, Boczkowski D, Moeller B, Dewhirst M, Vieweg J, and Gilboa E. Synergy between tumor immunotherapy and antiangiogenic therapy. Blood. 2003; 102(3):964-71.
67. Niethammer A G, Xiang R, Becker J C, Wodrich H, Pertl U, Karsten G, Eliceiri B P, and Reisfeld R A. A DNA vaccine against VEGF receptor 2 prevents effective angiogenesis and inhibits tumor growth. Nat Med. 2002; 8(12):1369-75.
68. Luo Y, Zhou H, Mizutani M, Mizutani N, Liu C, Xiang R, and Reisfeld R A. A DNA vaccine targeting Fos-related antigen 1 enhanced by IL-18 induces long-lived T-cell memory against tumor recurrence. Cancer Res. 2005; 65(8):3419-27.
69. Fassnacht M, Lee J, Milazzo C, Boczkowski D, Su Z, Nair S, and Gilboa E. Induction of CD4(+) and CD8(+) T-cell responses to the human stromal antigen, fibroblast activation protein: implication for cancer immunotherapy. Clin Cancer Res. 2005; 11(15):5566-71.
70. Lykke-Andersen S, and Jensen T H. Nonsense-mediated mRNA decay: an intricate machinery that shapes transcriptomes. Nature reviews Molecular cell biology. 2015; 16(11):665-77.
71. Bates P J, Laber D A, Miller D M, Thomas S D, and Trent J O. Discovery and development of the G-rich oligonucleotide AS1411 as a novel treatment for cancer. Exp Mol Pathol. 2009; 86(3):151-64.
72. Anandasabapathy N, Feder R, Mollah S, Tse S W, Longhi M P, Mehandru S, Matos I, Cheong C, Ruane D, Brane L, et al. Classical Flt3L-dependent dendritic cells control immunity to protein vaccine. J Exp Med. 2014; 211(9):1875-91.
73. Okano F, Merad M, Furumoto K, and Engleman E G. In vivo manipulation of dendritic cells overcomes tolerance to unmodified tumor-associated self antigens and induces potent anti-tumor immunity. J Immunol. 2005; 174(5):2645-52.
74. Salmon H, Idoyaga J, Rahman A, Leboeuf M, Remark R, Jordan S, Casanova-Acebes M, Khudoynazarova M, Agudo J, Tung N, et al. Expansion and Activation of CD103(+) Dendritic Cell Progenitors at the Tumor Site Enhances Tumor Responses to Therapeutic PD-L1 and BRAF Inhibition. Immunity. 2016; 44(4):924-38.
75. Spitzer M H, Carmi Y, Reticker-Flynn N E, Kwek S S, Madhireddy D, Martins M M, Gherardini P F, Prestwood T R, Chabon J, Bendall S C, et al. Systemic Immunity Is Required for Effective Cancer Immunotherapy. Cell. 2017, 168:487-502.
76. Gupta S, Clark E S, Termini J M, Boucher J, Kanagavelu S, LeBranche C C, Abraham S, Montefiori D C, Khan W N, and Stone G W. DNA vaccine molecular adjuvants SP-D-BAFF and SP-D-APRIL enhance anti-gp120 immune response and increase HIV-1 neutralizing antibody titers. J Virol. 2015; 89(8):4158-69.
77. Corry D B, and Kheradmand F. Induction and regulation of the IgE response. Nature. 1999; 402(6760 Suppl):B18-23.
78. Agrawal S, and Kandimalla E R. Role of Toll-like receptors in antisense and siRNA [corrected]. Nat Biotechnol. 2004; 22(12):1533-7.
79. Akira S, Uematsu S, and Takeuchi O. Pathogen recognition and innate immunity. Cell. 2006; 124(4):783-801.
80. Judge A D, Sood V, Shaw J R, Fang D, McClintock K, and MacLachlan I. Sequence-dependent stimulation of the mammalian innate immune response by synthetic siRNA. Nat Biotechnol. 2005; 23(4):457-62.
81. Gilboa E. The makings of a tumor rejection antigen. Immunity. 1999; 11(3):263-70.
82. Schumacher T N, and Schreiber R D. Neoantigens in cancer immunotherapy. Science. 2015; 348(6230):69-74.
83. Stronen E, Toebes M, Kelderman S, van Buuren M M, Yang W, van Rooij N, Donia M, Boschen M L, Lund-Johansen F, Olweus J, et al. Targeting of cancer neoantigens with donor-derived T cell receptor repertoires. Science. 2016; 352(6291):1337-41.
84. Schietinger A, Philip M, Krisnawan V E, Chiu E Y, Delrow J J, Basom R S, Lauer P, Brockstedt D G, Knoblaugh S E, Hammerling G J, et al. Tumor-Specific T Cell Dysfunction Is a Dynamic Antigen-Driven Differentiation Program Initiated Early during Tumorigenesis. Immunity. 2016; 45(2):389-401.
85. Gajewski T F, Schreiber H, and Fu Y X. Innate and adaptive immune cells in the tumor microenvironment. Nat Immunol. 2013; 14(10):1014-22.
86. Spranger S, Luke J J, Bao R, Zha Y, Hernandez K M, Li Y, Gajewski A P, Andrade J, and Gajewski T F. Density of immunogenic antigens does not explain the presence or absence of the T-cell-inflamed tumor microenvironment in melanoma. Proc Nati Acad Sci USA. 2016; 113(48):E7759-E68.
87. Spranger S, Bao R, and Gajewski T F. Melanoma-intrinsic beta-catenin signalling prevents anti-tumour immunity. Nature. 2015; 523(7559):231-5.
88. Corrales L, Glickman L H, McWhirter S M, Kanne D B, Sivick K E, Katibah G E, Woo S R, Lemmens E, Banda T, Leong J J, et al. Direct Activation of STING in the Tumor Microenvironment Leads to Potent and Systemic Tumor Regression and Immunity. Cell reports. 2015; 11(7):1018-30.
89. Liu K, and Nussenzweig M C. Origin and development of dendritic cells. Immuno) Rev. 2010; 234(1):45-54.
90. Callahan M K, Postow M A, and Wolchok J D. Targeting T Cell Co-receptors for Cancer Therapy. Immunity. 2016; 44(5):1069-78.
91. Gangadhar T C, and Vonderheide R H. Mitigating the toxic effects of anticancer immunotherapy. Nat Rev Clin Oncol. 2015; 11(91-9.
92. Boutros C, Tarhini A, Routier E, Lambotte O, Ladurie F L, Carbonnel F, Izzeddine H, Marabelle A, Champiat S, Berdelou A, et al. Safety profiles of anti-CTLA-4 and anti-PD-1 antibodies alone and in combination. Nature reviews Clinical oncology. 2016.
93. Beck K E, Blansfield J A, Tran K Q, Feldman A L, Hughes M S, Royal R E, Kammula U S, Topalian S L, Sherry R M, Kleiner D, et al. Enterocolitis in patients with cancer after antibody blockade of cytotoxic T-lymphocyte-associated antigen 4. J Clin Oncol. 2006; 24(15):2283-9.
94. Wang C, Lin G H, McPherson A J, and Watts T H. Immune regulation by 4-1BB and 4-1BBL: complexities and challenges. Immunol Rev. 2009; 229(1):192-215.
95. Dubrot J, Milheiro F, Alfaro C, Palazon A, Martinez-Forero I, Perez-Gracia J L, Morales-Kastresana A, Romero-Trevejo J L, Ochoa M C, Hervas-Stubbs S, et al. Treatment with anti-CD137 mAbs causes intense accumulations of liver T cells without selective anti-tumor immunotherapeutic effects in this organ. Cancer Immunol Immunother. 2010; 59(8):1223-33.
96. Niu L, Strahotin S, Hewes B, Zhang B, Zhang Y, Archer D, Spencer T, Dillehay D, Kwon B, Chen L, et al. Cytokine-mediated disruption of lymphocyte trafficking, hemopoiesis, and induction of lymphopenia, anemia, and thrombocytopenia in anti-CD137-treated mice. J Immunol. 2007; 178(7):4194-213.
97. Yonezawa A, Dutt S, Chester C, Kim J, and Kohrt H E. Boosting Cancer Immunotherapy with Anti-CD137 Antibody Therapy. Clin Cancer Res. 2015; 21(14):3113-20.
98. Macher B A, and Galili U. The Galalpha1,3Galbeta1,4GlcNAc-R (alpha-Gal) epitope: a carbohydrate of unique evolution and clinical relevance. Biochim Biophys Acta. 2008; 1780(2):75-88.
99. Thall A D, Maly P, and Lowe J B. Oocyte Gal alpha 1,3Gal epitopes implicated in sperm adhesion to the zona pellucida glycoprotein ZP3 are not required for fertilization in the mouse. J Biol Chem. 1995; 270(37):21437-40.
100. LaTemple D C, and Galili U. Adult and neonatal anti-Gal response in knock-out mice for alpha1,3galactosyltransferase. Xenotransplantation. 1998; 5(3):191-6.
101. LaTemple D C, Abrams J T, Zhang S Y, and Galili U. Increased immunogenicity of tumor vaccines complexed with anti-Gal: studies in knockout mice for alpha1,3galactosyltransferase. Cancer Res. 1999; 59(14):3417-23.
102. Gorelik E, Duty L, Anaraki F, and Galili U. Alterations of cell surface carbohydrates and inhibition of metastatic property of murine melanomas by alpha 1,3 galactosyltransferase gene transfection. Cancer Res. 1995; 55(18):4168-73.
103. Galili U, Wigglesworth K, and Abdel-Motal U M. Intratumoral injection of alpha-gal glycolipids induces xenograft-like destruction and conversion of lesions into endogenous vaccines. J Immunol. 2007; 178(7):4676-87.
104. Uchida J, Hamaguchi Y, Oliver J A, Ravetch J V, Poe J C, Haas K M, and Tedder T F. The innate mononuclear phagocyte network depletes B lymphocytes through Fc receptor-dependent mechanisms during anti-CD20 antibody immunotherapy. J Exp Med. 2004; 199(12):1659-69.
105. Musolino A, Naldi N, Bortesi B, Pezzuolo D, Capelletti M, Missale G, Laccabue D, Zerbini A, Camisa R, Bisagni G, et al. Immunoglobulin G fragment C receptor polymorphisms and clinical efficacy of trastuzumab-based therapy in patients with HER-2/neu-positive metastatic breast cancer. J Clin Oncol. 2008; 26(11):1789-96.
106. Bibeau F, Lopez-Crapez E, Di Fiore F, Thezenas S, Ychou M, Blanchard F, Lamy A, Penault-Llorca F, Frebourg T, Michel P, et al. Impact of Fc{gamma}RIIa-Fc{gamma}RIIIa polymorphisms and KRAS mutations on the clinical outcome of patients with metastatic colorectal cancer treated with cetuximab plus irinotecan. J Clin Oncol. 2009; 27(7):1122-9.
107. Selby M J, Engelhardt J J, Quigley M, Henning K A, Chen T, Srinivasan M, and Korman A J. Anti-CTLA-4 antibodies of IgG2a isotype enhance antitumor activity through reduction of intratumoral regulatory T cells. Cancer immunology research. 2013; 1(1):32-42.
108. Simpson T R, Li F, Montalvo-Ortiz W, Sepulveda M A, Bergerhoff K, Arce F, Roddie C, Henry J Y, Yagita H, Wolchok J D, et al. Fc-dependent depletion of tumor-infiltrating regulatory T cells co-defines the efficacy of anti-CTLA-4 therapy against melanoma. J Exp Med. 2013; 210(9):1695-710.
109. Bulliard Y, Jolicoeur R, Windman M, Rue S M, Ettenberg S, Knee D A, Wilson N S, Dranoff G, and Brogdon J L. Activating Fc gamma receptors contribute to the antitumor activities of immunoregulatory receptor-targeting antibodies. J Exp Med. 2013; 210(9):1685-93.
110. Dahan R, Sega E, Engelhardt J, Selby M, Korman A J, and Ravetch J V. FcgammaRs Modulate the Anti-tumor Activity of Antibodies Targeting the PD-1/PD-L1 Axis. Cancer Cell. 2015; 28(3):285-95.
111. Callahan M K, Postow M A, and Wolchok J D. Targeting T Cell Co-receptors for Cancer Therapy. Immunity. 2016; 44(5):1069-78.
112. Postow M A, Callahan M K, and Wolchok J D. Immune Checkpoint Blockade in Cancer Therapy. J Clin Oncol. 2015; 33(17):1974-82.
113. Sharma P, and Allison J P. Immune checkpoint targeting in cancer therapy: toward combination strategies with curative potential. Cell 2015; 161(2):205-14.
114. Topalian S L, Drake C G, and Pardoll D M. Immune checkpoint blockade: a common denominator approach to cancer therapy. Cancer Cell. 2015; 27(4):450-61.

1. A method of treating cancer in a subject in need thereof, comprising:
immunizing the subject against an antigen to generate an anti-antigen polyclonal antibody response; and
administering to the subject a conjugate comprising the antigen linked to a tumor targeting ligand.

2. The method of claim 1, wherein the antigen is a hapten.

3. The method of claim 2, wherein the hapten is selected from dinitrophenol (DNP), fluorescein, biotin, digoxigenin, digitoxigenin, gitoxigenin, strophanthidin, digoxin, digitoxin, ditoxin and strophanthin.

4. The method of claim 3, wherein the hapten is dinitrophenol (DNP).

5. A method of treating cancer in a subject in need thereof, comprising administering to the subject a conjugate comprising an antigen linked to a tumor targeting ligand,
wherein the antigen is recognized by naturally occurring polyclonal antibodies present in the subject.

6. The method of claim 5, wherein the antigen is Galα1-3Galβ1-4GlcNAc-R.

7. The method of claim 3, wherein the tumor targeting ligand recognizes one or more markers expressed on a tumor cell or the tumor environment.

8. The method of claim 7, wherein the tumor targeting ligand recognizes a marker associated with non-transformed tumor endothelial cells, products upregulated in the tumor stroma, or associated with the tumor vasculature.

9. The method of claim 8, wherein the tumor targeting ligand recognizes VEGF.

10. The method of claim 8, wherein the tumor targeting ligand recognizes osteopontin.

11. The method of claim 8, wherein the tumor targeting ligand recognizes one or more of immune checkpoint proteins.

12. The method of claim 11, wherein the immune checkpoint protein is selected from PD-1, PD-L1, PD-L2, and CTLA4.

13. The method of claim 3, wherein the tumor targeting ligand comprises an oligonucleotide.

14. The method of claim 13, wherein the oligonucleotide is an aptamer.

15. The method of claim 3, wherein the tumor targeting ligand comprises a protein-based targeting agent.

16. The method of claim 15, wherein the protein-based targeting agent is an antibody, an antibody derivative, or a peptide.

17. The method of claim 16, wherein the protein-based targeting agent is an antibody.

18. (canceled)

19. The method of claim 3, wherein the method is administered in combination with one or more therapies directed to:
increasing the neoantigenic content of the tumor cells,
blocking the function of the CD47 receptor,
enhancing intratumoral DC infiltration, promoting intratumor immune infiltration by downregulation of β-catenin,
promoting the survival and proliferation of tumor infiltrating T cells,
STING ligand administration, or
local sublethal irradiation.

20. The method of claim 3, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer; melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema, and Meigs' syndrome.

21. The method of claim 3, wherein the method
induces and/or enhances anti-tumor immune responses mediated by infiltrating dendritic cells;
induces and/or enhances anti-tumor responses mediated by CD4+ T cells;
induces and/or enhances humoral immune responses against tumors;
induces and/or enhances ADCC as mediated by Fc receptor (FcR)-expressing cells including dendritic cells, natural killer cells, macrophages, neutrophils, and eosinophils;
induces and/or enhances the destruction of immune suppressive cells; and/or
induces and/or enhances the destruction of tumor cells.
